(19) United States
(12) Patent Application Publication
(10) Pub. No.: US 2022/0339136 A1
(43) Pub. Date: Oct. 27, 2022

(54) LIPOSOMAL TROPONOID COMPOUND FORMULATIONS

(71) Applicant: United States Government As Represented By The Department of Veterans Affairs, Washington, DC (US)

(72) Inventors: Feng Cao, St. Louis, MO (US); Xinggou Cheng, San Antonio, TX (US)

(21) Appl. No.: 17/639,265

(22) PCT Filed: Aug. 28, 2020
(86) PCT No.: PCT/US2020/048358

§ 371 Date: Feb. 28, 2022

Related U.S. Application Data
(60) Provisional application No. 62/894,187, filed on Aug. 30, 2019.
(60) Provisional application No. 62/992,699, filed on Mar. 20, 2020.

(51) Int. Cl. A61K 31/265; A61K 9/127; A61K 31/122; A61K 31/381; A61K 31/192; A61K 31/235; A61K 31/185; A61K 31/575; A61K 31/355; A61P 31/04; A61K 31/7036; A61K 31/546

(52) U.S. Cl. CPC A61K 31/265; A61K 9/1271; A61K 31/122; A61K 31/381; A61K 31/192; A61K 31/235; A61K 31/185; A61K 31/575; A61K 31/355; A61P 31/04; A61K 31/7036; A61K 31/546

(57) ABSTRACT

The present disclosure is concerned with liposomal formulations comprising troponoid compounds and methods of using same in the treatment of, for example, viral infections, antimicrobial infections, cancer, inflammatory diseases, and cardiovascular diseases. This abstract is intended as a scanning tool for purposes of searching in the particular art and is not intended to be limiting of the present invention.

### CROSS-REFERENCE TO RELATED APPLICATIONS

This application claims the benefit of U.S. Provisional Application No. 62/894,187, filed on Aug. 30, 2019, and U.S. Provisional Application No. 62/992,699, filed on Mar. 20, 2020, the contents of which are incorporated herein by reference in their entireties.

### BACKGROUND

Tropolones can be found from natural plants such as western red cedar trees, theaflavins (antioxidant polyphenols from tea leaves) of black tea, etc. Chemically, tropolone has a structure shown in FIG. 1. Troponoid compounds include the tropones, tropolones, and hydroxytropolones (HT) and their derivatives. All of them (tropone, tropolone, HT and their derivatives) have a seven-carbon ring and possesses a nonbenzenoid aromatic character (Bentley, R., A fresh look at natural tropolonoids. Nat Prod Rep, 2008. 25(1): p. 118-38). Troponoids have high pharmacological activity and many of them are found to have antibacterial (Morita et al. (2004) Biological activity of beta-dolabrin, gamma-thujaplicin, and 4-acetyltropolone, hinokitiol-related compounds, Biol. Pharm. Bull. 27, 1666-9; Fotopoulou, T., et al., Antimicrobial/Antibiofilm Activity and Cytotoxic Studies of beta-Thujaplicin Derivatives. Arch Pharm (Weinheim), 2016. 349(9): p. 698-709; Arima, Y., Antibacterial effect of beta-thujaplicin on staphylococci isolated from atopic dermatitis: relationship between changes in the number of viable bacterial cells and clinical improvement in an eczematous lesion of atopic dermatitis. Journal of Antimicrobial Chemotherapy, 2002. 51(1): p. 113-122; Trust T J. (1975) Antibacterial activity of tropolone, Antimicrob Agents Chemother. 7, 500-6), antiviral (Ireland, P. J., et al., Synthetic alpha-Hydroxytropolones Inhibit Replication of Wild-Type and Acyclovir-Resistant Herpes Simplex Viruses. Antimicrob Agents Chemother, 2016. 60(4): p. 2140-9; Lu, G., et al., Hydroxylated Tropolones Inhibit Hepatitis B Virus Replication by Blocking the Viral Ribonuclease H Activity. Antimicrob Agents Chemother, 2015. 59(2): p. 1070-1079; Hu, Y., et al., beta-Thujaplicinol inhibits hepatitis B virus replication by blocking the viral ribonuclease H activity. Antiviral Res, 2013. 99(3): p. 221-9; Tavis, J. E., et al., The hepatitis B virus ribonuclease H is sensitive to inhibitors of the human immunodeficiency virus ribonuclease H and integrase enzymes. PLoS Pathog, 2013. 9(1): p. e1003125), antifungal properties (Donlin, M. J., et al., Troponoids Can Inhibit Growth of the Human Fungal Pathogen Cryptococcus neoformans. Antimicrob Agents Chemother, 2017. 61(4)), anti-tumor (Shih, Y. H., et al., In vitro antimicrobial and anticancer potential of hinokitiol against oral pathogens and oral cancer cell lines. Microbiol Res, 2013. 168(5): p. 254-62), anti-inflammatory, antioxidant and insecticidal properties (Zhao, J., Plant troponoids: chemistry, biological activity, and biosynthesis. Curr Med Chem, 2007. 14(24): p. 2597-621).

Two tropolone derivatives, compound nos. 62 and 285, were previously found to have antibacterial properties. Specifically, they can inhibit multidrug-resistant Staphylococcus. aureus (MDRSA), including methicillin-resistant S. aureus (MRSA) strains (Cao, F., et al., Synthesis and Evaluation of Troponoids as a New Class of Antibiotics. ACS Omega, 2018. 3(11): p. 15125-15133). S. aureus cause skin infection, pneumonia, septicemia, osteomyelitis, arthritis, endocarditis, and toxic shock syndrome (Tacconelli, E., M. Tumbarello, and R. Cauda. N Engl J Med. 1998; 339(27): 2026-7). If left untreated, S. aureus infections can become severe and cause sepsis with high mortality. Approximately 30% of the human population are asymptomatically and permanently colonized with nasal S. aureus (Sakr, A., et al., Staphylococcus aureus Nasal Colonization: An Update on Mechanisms, Epidemiology, Risk Factors, and Subsequent Infections. Front Microbiol, 2018. 9: p. 2419). MDRSA display decreased susceptibility to the current front-line antibiotics such as gentamicin, ciprofloxacin, vancomycin, daptomycin, and linezolid, etc (Perichon, B. and P. Courvalin, VanA-type vancomycin-resistant Staphylococcus aureus. Antimicrob Agents Chemother, 2009. 53(11): p. 4580-7; Nannini, E., B. E. Murray, and C. A. Arias, Resistance or decreased susceptibility to glycopeptides, daptomycin, and linezolid in methicillin-resistant Staphylococcus aureus. Curr Opin Pharmacol, 2010. 10(5): p. 516-21; Morales, G., et al., Resistance to linezolid is mediated by the cfr gene in the first report of an outbreak of linezolid-resistant Staphylococcus aureus. Clin Infect Dis, 2010. 50(6): p. 821-5). There is an urgent need to find new antibiotics with a novel scaffold to combat the growing problem of antibiotic resistance for S. aureus infections. Compound 62 and 285 has been shown the effective inhibition on MDRSA, based on our previous study (Cao, F., et al., Synthesis and Evaluation of Troponoids as a New Class of Antibiotics. ACS Omega, 2018. 3(11): p. 15125-15133).

Unfortunately, the development of soluble, absorbale forms of troponoid compounds for the treatment of diseases has remained elusive. Troponoid compound 62 and 285 are not quite soluble in water, but can be dissolved in an organic polar solvent called Dimethyl sulfoxide (DMSO). However, it is known that DMSO can induce reported side effects including headaches, burning and itching on contact with the skin, and strong allergic reactions (Hanslick, J. L., et al., Dimethyl sulfoxide (DMSO) produces widespread apoptosis in the developing central nervous system. Neurobiol Dis, 2009. 34(1): p. 1-10). DMSO is also a known developmental neurotoxin which may cause brain degeneration at level as low as 0.3 mL/kg. Troponoid compound 62 or 285 may also be dissolved used organic solvent such as chloroform or a chloroform and methanol mixture (e.g., 1:1 v:v ratio). However, it is known that chloroform and methanol are toxic organic solvent. For compounds 62 and 285 and other hydrophobic troponoid compound, a novel strategy is needed to make them into acceptable drug products for clinical use. In addition, for hydrophilic troponoid compounds that can be dissolved in water, an alternative formulation is needed for sustained and/or targeted delivery. Thus, there remains a need for formulations of troponoid compounds and methods of making and using same.

### SUMMARY

In accordance with the purpose(s) of the invention, as embodied and broadly described herein, the invention, in one aspect, relates to liposomal troponoid compound formulations compositions and methods of using same in the treatment of diseases and disorders such as, for example, viral infections, antimicrobial infections, cancer, inflammatory diseases, and cardiovascular diseases.

Disclosed are liposome formulations comprising a troponoid having a structure represented by a formula:

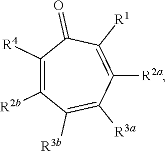

wherein R¹ is selected from hydrogen, halogen, —OH, —SH, —OC(O)Ar¹, —SC(O)Ar¹, —OC(O)(C1-C4 alkyl)Ar¹, —SC(O)(C1-C4 alkyl)Ar¹, and —OSO₂Ar¹; wherein Ar¹, when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a} and R^{2b} is independently selected from hydrogen, halogen, —OH, —CO₂H, and Ar²; wherein Ar², when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{3a} and R^{3b} is independently selected from hydrogen, halogen, C1-C6 alkyl, —C(O)R¹¹, —C(O)Ar³, and Ar³; wherein R¹¹, when present, is selected from C1-C4 alkyl and C3-C6 cycloalkyl; wherein Ar³, when present, is selected from C2-C5 heteroaryl and C6-C12 aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R⁴ is selected from hydrogen, halogen, and —OH, or a pharmaceutically acceptable salt thereof.

Also disclosed are liposome formulations comprising a troponoid, a lipid, and vitamin E.

Also disclosed are liposome formulations comprising: (a) a troponoid in an amount of about 15 wt % or less, wherein the troponoid is selected from:

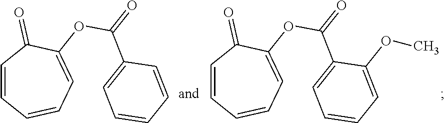

phosphatidylcholine in an amount of from about 60 wt % to about 99 wt %; cholesterol in an amount of about 20 wt % or less; and vitamin E in an amount of about 17 wt % or less.

Also disclosed are nanoparticles comprising a disclosed formulation.

Also disclosed are methods for treating a disease or disorder in a subject, the method comprising administering to the subject an effective amount of a disclosed formulation, wherein the disease or disorder is a viral infection, an antimicrobial infection, cancer, an inflammatory disease, or a cardiovascular disease.

Also disclosed are kits comprising a disclosed formulation, and one or more of: (a) an agent selected from antibiotic agent, an antibiotic agent, an antibacterial agent, an antifungal agent, an antiviral agent, a chemotherapeutic agent, an anti-inflammatory agent, and a cardiac agent; and (b) instructions for treating a viral infection, an antimicrobial infection, cancer, an inflammatory disease, and/or a cardiovascular disease.

Also disclosed are methods of making a liposomal formulation, the method comprising: (a) providing a lipid solution comprising one or more lipids and an organic solvent; (b) removing the solvent, thereby forming a lipid cake; (c) mixing the lipid cake with a hydration media, thereby forming a hydrated solution; and (d) extruding the hydrated solution, thereby forming a liposomal formulation, wherein either the lipid solution or the hydration media comprises a troponoid.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows a representative structures of troponoid compounds, including the structures of tropone (A), tropolone (B), α-hydroxytropolone (α-HT) (C), compound no. 62 (D), and compound no. 285 (E).

FIG. 2 shows representative images illustrating a comparison of compound no. 285 (1), liposomal compound no. 285 (2), compound no. 62 (3), liposomal compound no. 62 (4), liposomal gentamicin (5), liposomal ceftriaxone (6), 2% mupirocin ointment (7), and liposomal H₂O (8) on the inhibition of WT S. aureus (Sa25923) and MDRSA AR219 and AR228 strains.

FIG. 3 shows a representative schematic of the infection and treatment in a nasal bacteria colonization model.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form, which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, and amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions that can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms, and may also contain one or more heteroatoms. Suitable heteroatoms include, but are not limited to, O, N, Si, P and S, wherein the nitrogen, phosphorous and sulfur atoms are optionally oxidized, and the nitrogen heteroatom is optionally quaternized. Aliphatic groups include, but are not limited to, linear or branched, alkyl, heteroalkyl, alkenyl, heteroalkenyl, alkynyl, and heteroalkynyl groups, and hybrids thereof such as (cycloalkyl)alkyl, (heterocycloalkyl)alkyl, (cycloalkenyl)alkyl, (heterocycloalkyenyl)alkyl, (cycloalkyl)alkenyl, or (cycloalkyl)heteroalkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a non-aromatic carbon-based ring type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹—OA² or —OA¹-(OA²)ₐ—OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

If a group contains multiple ring structures in which, for example, one aryl group and one cycloalkyl group are bound together via a fused ring structure, such a group will generally be referred to as a cycloalkyl (or, in the case of one aryl or heteroaryl group and one cycloalkyl or heterocycloalkyl group, a heterocycloalkyl group), as the entire ring structure is not aromatic. Alternatively, such a group may be referred to as an aryl (or, when the aromatic ring contains one or more heteroatoms, heteroaryl) group having two substituents covalently bound to form a ring.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula -NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, 0, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group that has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula -SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “R,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R*, -(haloR^{●}), —OH, —OR^{●}, —O(haloR^{●}), —CN, —C(O)OH, —C(O)OR^{●}, —NH₂, —NHR^{●}, —NR^{●}2, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}2, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

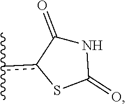

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radioactively labeled forms, isomers, solvates and combinations thereof. The “combinations” mentioned in this context are refer to derivatives falling within at least two of the groups: pharmaceutically acceptable salts, prodrugs, deuterated forms, radioactively labeled forms, isomers, and solvates. Examples of radioactively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically labeled or isotopically substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, 13C, ¹⁴C, ¹⁵N, ¹⁸O, ¹⁷O, 35S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., 3H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules that owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

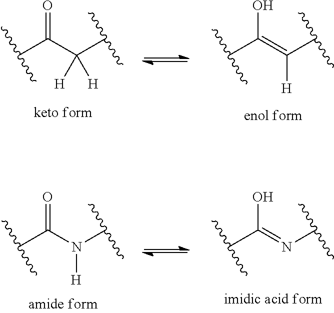 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

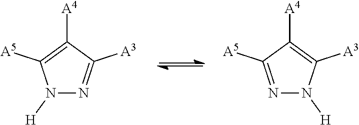

Unless stated to the contrary, the invention includes all such possible tautomers.

It is known that chemical substances form solids that are present in different states of order that are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

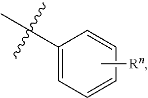

which is understood to be equivalent to a formula:

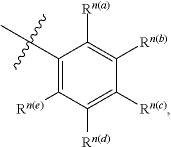

wherein n is typically an integer. That is, R is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, Mass.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. LIPOSOME FORMULATIONS

In one aspect, disclosed are liposome formulations comprising a troponoid and a lipid. Thus, in various aspects, disclosed are liposome formulations comprising a troponoid having a structure represented by a formula:

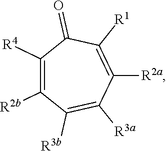

wherein R¹ is selected from hydrogen, halogen, —OH, —SH, —OC(O)Ar¹, —SC(O)Ar¹, —OC(O)(C1-C4 alkyl)Ar¹, —SC(O)(C1-C4 alkyl)Ar¹, and —OSO₂Ar¹; wherein Ar¹, when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a} and R^{2b} is independently selected from hydrogen, halogen, —OH, —CO₂H, and Ar²; wherein Ar², when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{3a} and R^{3b} is independently selected from hydrogen, halogen, C1-C6 alkyl, —C(O)R¹, —C(O)Ar³, and Ar³; wherein R¹¹, when present, is selected from C1-C4 alkyl and C3-C6 cycloalkyl; wherein Ar³, when present, is selected from C2-C5 heteroaryl and C6-C12 aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R⁴ is selected from hydrogen, halogen, and —OH, or a pharmaceutically acceptable salt thereof.

In one aspect, disclosed are liposome formulations comprising a troponoid, a lipid, and vitamin E.

In one aspect, disclosed are liposome formulations comprising: (a) a troponoid in an amount of about 15 wt % or less, wherein the troponoid is selected from:

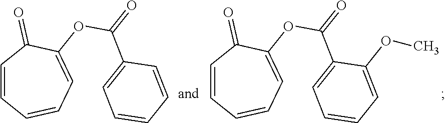

phosphatidylcholine in an amount of from about 60 wt % to about 99 wt %; cholesterol in an amount of about 20 wt % or less; and vitamin E in an amount of about 17 wt % or less.

In one aspect, disclosed are liposome formulations comprising phosphatidylcholine present in the range of 60 wt % to 99 wt %, cholesterol present in the range of 0 wt % to 20 wt %, vitamin-E present in the range of 0 wt % to 17 wt %, and a troponoid compound present in the range of 0 wt % to 15 wt %, wherein said formulation is selected to equal 100 wt %.

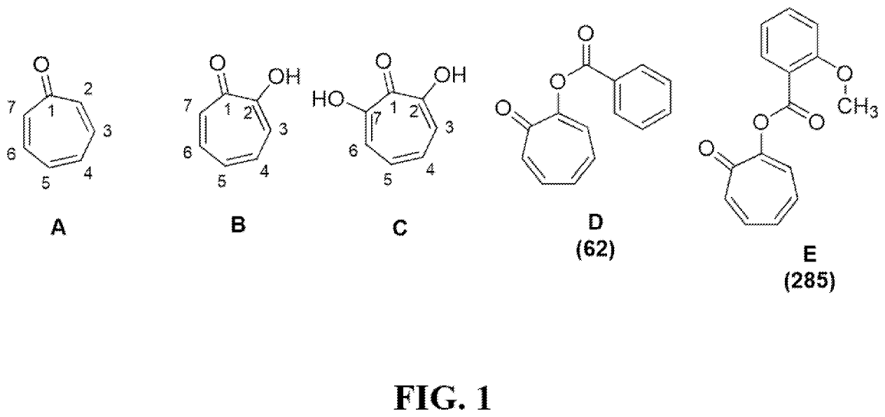

In various aspects, the present disclosure is directed to liposomal troponoid compound formulations preferably including one or more of tropone, tropolone, and/or hydroxytropolone (HT) compounds and their derivatives such as those shown containing structure similar to those in FIG. 1. Such troponoid compounds may be utilized for treatment of infection due to bacteria, fungi, or virus, as well as for the treatment of other conditions, including, but not limited to, wound healing, cancer, inflammation, or as free-radical scavengers. The formulations may particularly include liquid formulations or lyophilized powder that may be reconstituted, or in the form of a cream or ointment. Various troponoid compounds may be employed for use herein, both hydrophobic and hydrophilic, and particularly, compound 62 and compound 285 as shown in FIG. 1.

Without wishing to be bound by theory, the term “hydrophobic troponoids,” as used herein, refers to troponoid compounds that are insoluble in water. For example, a hydrophobic troponoid can be soluble in water in an amount of 10 mg/l or less, 9 mg/l or less, 8 mg/l or less, 7 mg/l or less, 6 mg/l or less, 5 mg/l or less, 4 mg/l or less, 3 mg/l or less, 2 mg/l or less, or 1 mg/l or less. Solubility of less than 10 mg/l may be considered as having relatively low solubility and hydrophobic. In various further aspects, the disclosed hydrophobic troponoids can be at least about 10%, at least about 20%, at least about 30%, at least about 40%, at least about 50%, at least about 60%, at least about 70%, at least about 80%, at least about 90%, or at least about 95% insoluble in water. Compound nos. 62 and 285 are available as a powder and are reportedly insoluble in water as defined herein. Thus, these compounds are considered herein as hydrophobic troponoids.

Without wishing to be bound by theory, the term “hydrophilic troponoids,” as used herein, refers to troponoid compounds that are soluble in water. For example, a hydrophobic troponoid can be soluble in water in an amount of 10 mg/l or higher, 15 mg/l or higher, 20 mg/l or higher, 25 mg/l or higher, 30 mg/l or higher, 35 mg/l or higher, 40 mg/l or higher, 45 mg/l or higher, 50 mg/l or higher, 55 mg/l or higher, 60 mg/l or higher, 65 mg/l or higher, 70 mg/l or higher, or 75 mg/l or higher. Solubility herein is that solubility determined at a temperature in the range of from about 20° C. to about 25° C. In various further aspects, the disclosed hydrophobic troponoids can be at least about 10%, at least about 20%, at least about 30%, at least about 40%, at least about 50%, at least about 60%, at least about 70%, at least about 80%, at least about 90%, or at least about 95% soluble in water.

In the disclosed compositions and methods, either hydrophilic troponoids or hydrophobic troponoids or both can be incorporated into a liposomal troponoid formulation. The liposomal formulation preferably includes phosphatidylcholine, cholesterol, and vitamin-E as lipids. The phosphatidylcholine is preferably sourced form soybeans or egg yolk, or preferably other sources. In embodiments, the liposomal troponoid compound formulation may be available as a powder. The powder may be reconstituted to provide the troponoid formulation in aqueous media. The phosphatidylcholine may be present in in the range of 60 wt % to 99 wt %, cholesterol present in the range of 0 wt % to 20 wt %, vitamin-E present in the range of 0 wt % to 17 wt % including all values and ranges therein, said troponoid compound present in the range of 0 wt % to 15 wt % including all values and ranges therein, wherein said formulation is selected to equal 100 wt % (not considering the water, solvent, or cryoprotective agents).

Further, the liposome solution may include optionally cryo-protective agent, such as sucrose or trehalose, present in the range of 0 wt % to 15 wt % of the total weight of the solution, including all values and ranges therein and preferably in the range of 5 wt % to 15 wt %. The remainder of the solution includes aqueous media.

Optionally, the incorporation of other hydrophilic or hydrophobic drugs or compositions into the formulation can be considered. For example, one may incorporate gentamicin or Ceftriaxone or polypeptides.

In various aspects, the troponoid is hydrophilic. In various further aspects, the troponoid is hydrophobic.

In various aspects, the troponoid is non-naturally occurring.

In various aspects, the troponoid has a structure represented by a formula:

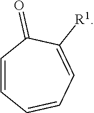

In various aspects, the troponoid has a structure represented by a formula:

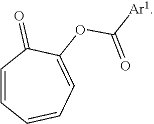

In various aspects, the troponoid is selected from:

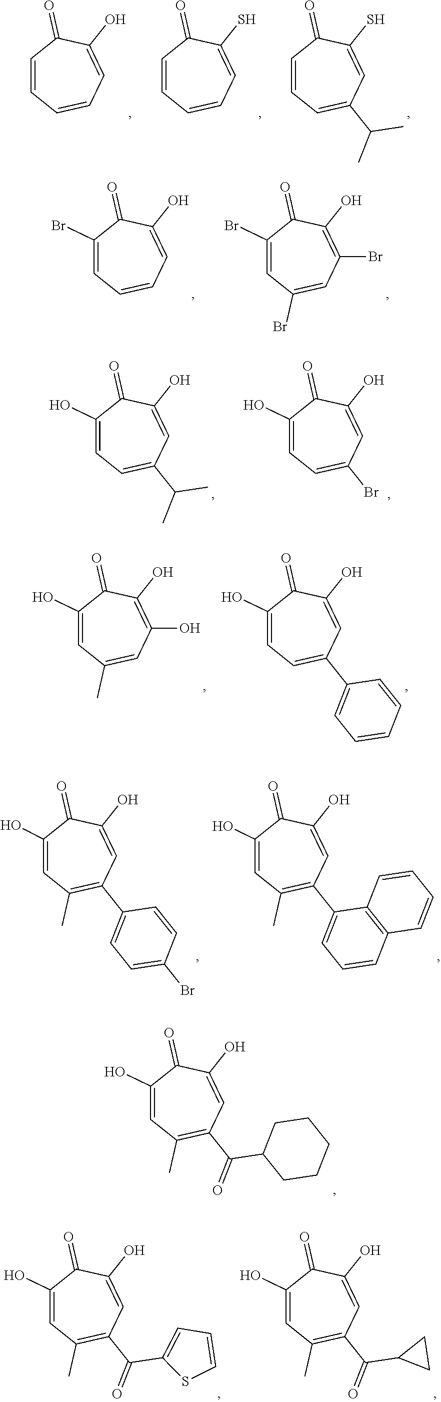

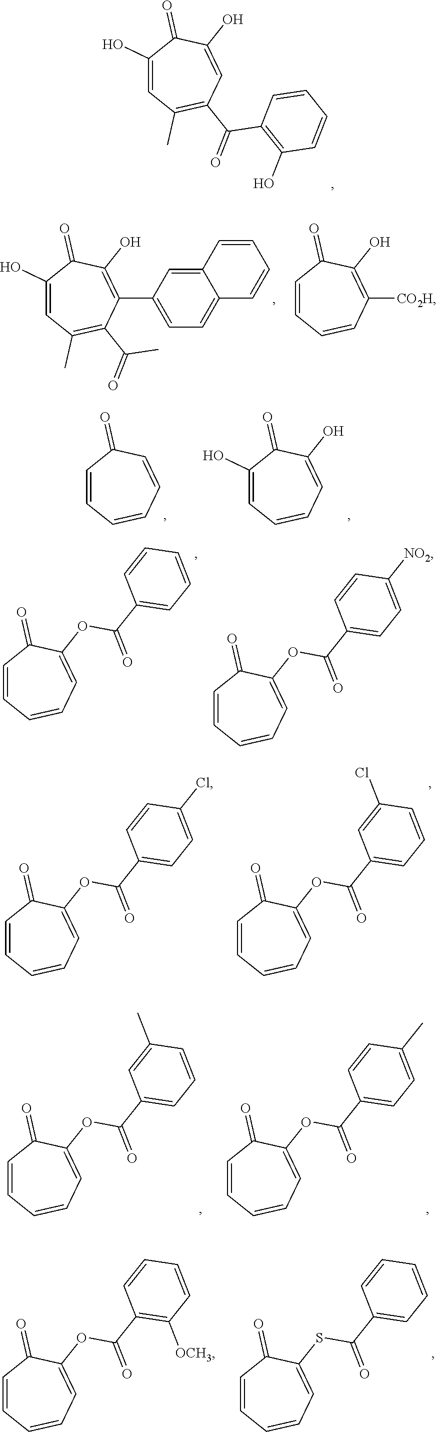

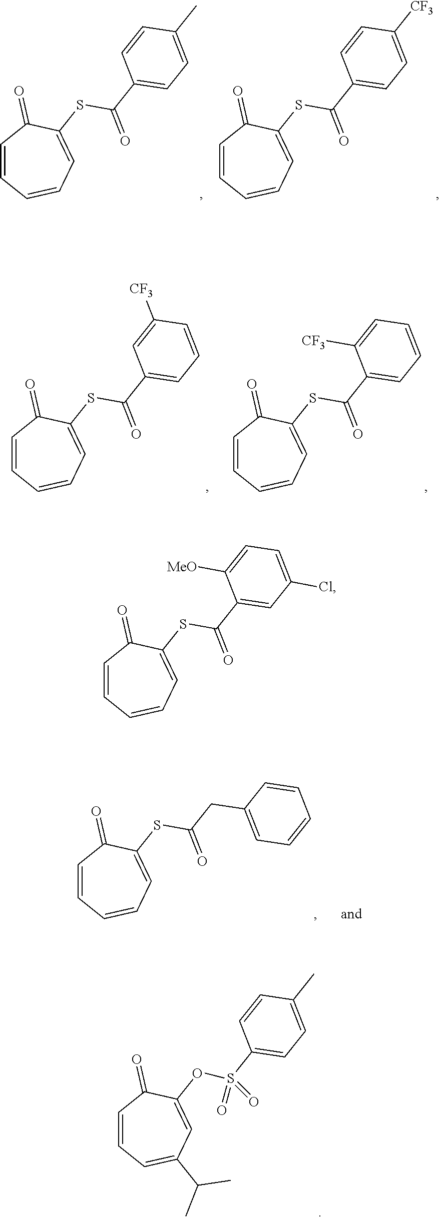

In various aspects, the troponoid is selected from:

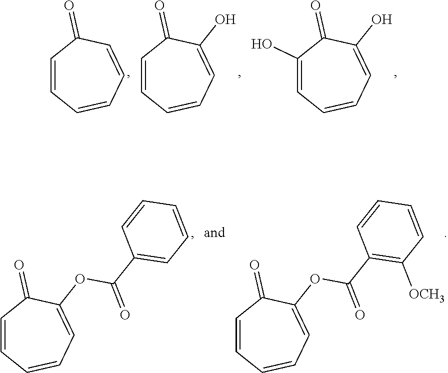

In various aspects, the troponoid is selected from:

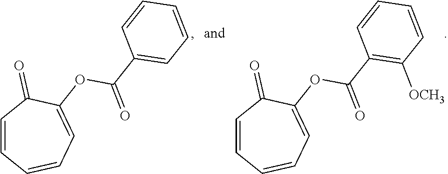 [SMILES: O=C(Oc1cccccc1=O)c1ccccc1 ; COc1ccccc1C(=O)Oc1cccccc1=O]

In various aspects, the troponoid is present in an amount of about 15 wt % or less, based on the weight of the formulation. Thus, in various further aspects, the troponoid is present in an amount of about 30 wt % or less, 25 wt % or less, 20 wt % or less, 15 wt % or less, 10 wt % or less, 5 wt % or less, or 1 wt % or less, based on the weight of the formulation.

In various aspects, the liposome formulation comprises an effective amount of the troponoid compound. In a further aspect, an effective amount is a therapeutically effective amount. In a still further aspect, an effective amount is a prophylactically effective amount.

In various aspects, the liposome formulation comprises a lipid. Examples of lipids include, but are not limited to, phosphatidylcholine (PC), including but not limited to natural PC (soy PC or egg Yok/heart/liver/brain PC) or synthetic PC such as X:0 PC, where X refers to the carbon number of saturated and/or unsaturated fatty acid, ranging from 3 to 24. Typical synthetic lipid examples include, but are not limited to, 1,2-dimyristoyl-sn-glycero-3-phosphocholine (DMPC), 1,2-dipalmitoyl-sn-glycero-3-phosphocholine (DPPC), and 1,2-distearoyl-sn-glycero-3-phosphocholine (DSPC).

In various aspects, the lipid is present in an amount of from about 60 w % to about 99 wt %, based on the weight of the formulation. Thus, in various aspects, the lipid is present in an amount of from about 50 wt % to about 99 wt %, 60 wt % to about 99 wt %, 70 wt % to about 99 wt %, 80 wt % to about 99 wt %, 90 wt % to about 99 wt %, 60 wt % to about 90 wt %, 60 wt % to about 80 wt %, or 60 wt % to about 70 wt %, based on the weight of the formulation.

In various aspects, the lipid is sourced from soybeans or egg yolk. In a further aspect, the lipid is sourced from soybeans. In a still further aspect, the lipid is sourced from egg yolk.

In various aspects, the liposome formulation further comprises cholesterol. The cholesterol can be present in an amount of about 30 wt % or less, 25 wt % or less, 20 wt % or less, 15 wt % or less, 10 w % or less, or 5 wt % or less, based on the weight of the formulation.

In various aspects, the liposome formulation further comprises vitamin E. The vitamin E can be present in an amount of about 17 wt % or less, based on the weight of the formulation. Thus, in various further aspects, the vitamin E can be present in an amount of about 25 wt % or less, 23 wt % or less, 20 wt % or less, 17 wt % or less, 15 wt % or less, 12 wt % or less, 12 wt % or less, 10 wt % or less, 8 wt % or less, 6 wt % or less, 5 wt % or less, 4 wt % or less, 3 wt % or less, 2 wt % or less, or 1 wt % or less, based on the weight of the formulation.

In various aspects, the liposome formulation further comprises a cryo-protective agent. Examples of cryo-protective agents include, but are not limited to, sucrose and trehalose. The cryo-protective agent can be present in an amount of about 15 wt % or less, based on the weight of the formulation. Thus, in various further aspects, the cryo-protective agent can be present in an amount of about 25 wt % or less, 20 wt % or less, 15 wt % or less, 10 wt % or less, or 5 wt % or less, based on the weight of the formulation. In a still further aspect, the cryo-protective agent can be present in an amount of from about 5 wt % to about 25 wt %, from about 5 wt % to about 20 wt %, from about 5 wt % to about 15 wt %, from about 5 wt % to about 10 wt %, from about 10 wt % to about 25 wt %, from about 15 wt % to about 25 wt %, from about 20 wt % to about 25 wt %, or from about 5 wt % to about 15 wt %, based on the weight of the formulation.

In various aspects, the liposome formulation further comprises an antibiotic agents. Examples of antibiotic agents include, but are not limited to, lipopeptides, fluoroquinolone, lipoglycopeptides, macrolides, β-lactams such as penicillins, cephalosporins, monobactams, and carbapenems, lincosamides, streptogramins, aminoglycosides, quinolones, sulfonamides, tetracyclines, chloramphenicol, metronidazole, tinidazole, nitrofurantoin, glycopeptides, lipoglycopeptides, oxazolidinones, rifamycins, polypeptides, and tuberactinomycins. In a further aspect, the antibiotic agent is selected from gentamicin and ceftriaxone.

In various aspects, the liposome formulation further comprises an antibacterial agent. Examples of antibacterial agents include, but are not limited to, amoxicillin, ampicillin, azithromycin, aztreonam, azlocillin, bacitracin, carbenicillin, cefaclor, cefadroxil, cefamandole, cefazolin, cephalexin, cefdinir, cefditorin, cefepime, cefixime, cefoperazone, cefotaxime, cefoxitin, cefpodoxime, cefprozil, ceftazidime, ceftibuten, ceftizoxime, ceftriaxone, cefuroxime, chloramphenicol, cilastin, ciprofloxacin, clarithromycin, clavulanic acid, clinafloxacin, clindamycin, clofazimine, cloxacillin, colistin, dalbavancin, dalfopristin, demeclocycline, dicloxacillin, dirithromycin, doxycycline, erythromycin, enrofloxacin, enoxacin, enviomycin, ertepenem, ethambutol, flucloxacillin, fosfomycin, furazolidone, gatifloxacin, gentamicin, imipenem, isoniazid, kanamycin, linezolid, lomefloxacin, loracarbef, mafenide, moxifloxacin, meropenem, metronidazole, mezlocillin, minocycline, mupirocin, nafcillin, nalidixic acid, neomycin, netilmicin, nitrofurantoin, norfloxacin, ofloxacin, oritavancin, oxytetracycline, penicillin, piperacillin, platensimycin, polymixin B, quinupristin, retapamulin, rifabutin, rifampin, rifapentine, roxithromycin, sparfloxacin, spectinomycin, sulbactam, sulfacetamide, sulfamethizole, sulfamethoxazole, teicoplanin, telithromycin, telavancin, temafloxacin, tetracycline, thioacetazone, thioridazine, ticarcillin, tinidazole, tobramycin, torezolid, tosufloxacin, trimethoprim, troleandomycin, trovafloxacin, and vancomycin, or combinations thereof.

In various aspects, the liposome formulation further comprises an antifungal agent. Examples of antifungal agents include, but are not limited to, clotrimazole, econazole, miconazole, terbinafine, fluconazole, ketoconazole, and amphotericin.

In various aspects, the liposome formulation further comprises a stealth lipid. Examples of stealth lipids include, but are not limited to, 1,2-distearoyl-sn-glycero-3-phosphoethanolamine-N-[amino(polyethylene glycol) (DSPE-PEG), mPEG-DSPE, and 1,2-dioleoyl-sn-glycero-3-phosphoethanolamine-N-[amino(polyethylene glycol)] (DOPE), or pharmaceutically acceptable salts thereof.

In various aspects, the liposome formulation further comprises a positively charged molecule or a positively charged lipid. Examples of positively charged molecules include, but are not limited to, stearyl amines. Examples of positively charged lipids include, but are not limited to, 1,2-dimyristoyl-3-trimethylammonium-propane, 1,2-dilauroyl-sn-glycero-3-ethylphosphocholine, and pharmaceutically acceptable salts thereof.

In a further aspect, the formulation is formulated as a as a liquid, a lyophilized powder, a cream, or an ointment.

In a further aspect, the formulation is substantially free of dimethylsulfoxide (DMSO), methanol, and/or chloroform.

In a further aspect, the formulation is substantially free of organic solvent. Examples of organic solvents include, but are not limited to, methylene chloride, 1,1,1-trichloroethane, carbon tetrachloride, trichloroethylene, ethanol, n-hexane, methyl ethyl ketone, formaldehyde, acetone, benzene, toluene, and mixtures thereof.

In a further aspect, the formulation is administered to a mammal. In a still further aspect, the mammal is a human. In an even further aspect, the human is a patient.

In a further aspect, the formulation is used to treat a disease or disorder such as, for example, a viral infection, an antimicrobial infection, cancer, an inflammatory disease, or a cardiovascular disease.

It is understood that the disclosed formulations can be prepared from the disclosed troponoid compounds. It is also understood that the disclosed formulations can be employed in the disclosed methods of using.

### C. TROPONOID COMPOUNDS

In one aspect, the invention relates to troponoid compounds useful in treating viral infections, antimicrobial infections, cancer, inflammatory diseases, or cardiovascular diseases. Examples of troponoid compounds include, but are not hinted to, tropones, tropolones, and hydrocytropolones.

Tropolones can be extracted from natural plants such as western red cedar trees, theaflavins (antioxidant polyphenols from tea leaves) of black tea, etc. Chemically, tropolones have a structure shown in FIG. 1. Troponoid compounds include the tropones, tropolones, and hydroxytropolones (HT) and their derivatives. All of these compounds have a seven-carbon ring and possesses a non-benzenoid aromatic character. Troponoids have high pharmacological activity. Notably, for the last seven years Drs. Tavis and Morrison (PI at St. Louis University) have investigated the potential of tropolones in inhibition of hepatitis B virus (Lomonosova, E. et al. Efficacy and cytotoxicity in cell culture of novel alpha-hydroxytropolone inhibitors of hepatitis B virus ribonuclease H. Antiviral Res. 144, 164-172 (2017); Hu, Y., Cheng, X., Cao, F., Huang, A. & Tavis, J. E. beta-Thujaplicinol inhibits hepatitis B virus replication by blocking the viral ribonuclease H activity. Antiviral Res. 99, 221-229 (2013); Lu, G. et al. Hydroxylated tropolones inhibit hepatitis B virus replication by blocking viral ribonuclease H activity. Antimicrob. Agents Chemother. 59, 1070-1079 (2015)), Herpes Simplex Viruses (Ireland, P. J. et al. Synthetic alpha-Hydroxytropolones Inhibit Replication of Wild-Type and Acyclovir-Resistant Herpes Simplex Viruses. Antimicrob. Agents Chemother. 60, 2140-2149 (2016); Berkowitz, A. J. et al. Importance of lipophilicity for potent anti-herpes simplex virus-1 activity of alpha-hydroxytropolones. Medchemcomm 10, 1173-1176 (2019)), human fungal pathogen Cryptococcus neoformans (Donlin, M. J. et al. Troponoids Can Inhibit Growth of the Human Fungal Pathogen Cryptococcus neoformans. Antimicrob. Agents Chemother. 61, (2017)).

In various aspects, the troponoid is hydrophilic. In various further aspects, the troponoid is hydrophobic.

In various aspects, the troponoid is non-naturally occurring.

In one aspect, the disclosed troponoid compounds exhibit antimicrobial activity. Examples of antimicrobial infections include, but are not limited to, bacterial infections (e.g., M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neisseria meningitidis, Neisseria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species) and fungal infections (e.g., ringworm, a Candida infection, a fungal nail infection, Blastomyces infection, Cryptococcus gattii infection, Paracoccidioides infection, Coccidioides infection, and Histoplasmosis infection).

In one aspect, the disclosed troponoid compounds exhibit antiviral activity. Examples of viral infections include, but are not limited to, human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), human coronavirus, and zika (ZIKV).

In one aspect, the disclosed troponoid compounds exhibit chemotherapeutic activity. Examples of cancers include, but are not limited to, a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma).

In one aspect, the disclosed troponoid compounds exhibit anti-inflammatory activity. Examples of inflammatory diseases include, but are not limited to, rheumatoid arthritis, insulin-dependent diabetes mellitus, multiple sclerosis, myasthenia gravis, Crohn's disease, autoimmune nephritis, primary biliary cirrhosis, psoriasis, acute pancreatitis, allograph rejection, allergic inflammation, inflammatory bowel disease, septic shock, osteoporosis, osteoarthritis, and cognitive deficits induced by neuronal inflammation.

In one aspect, the disclosed troponoid compounds exhibit cardiovascular activity. Examples of cardiovascular diseases include, but are not hinted to, heart failure and myocardial infarction.

In one aspect, the troponoid compounds of the invention are useful in the treatment of antimicrobial infections, viral infections, cancer, inflammatory diseases, and/or cardiovascular diseases, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Structure

In one aspect, disclosed are troponoid compounds having a structure represented by a formula:

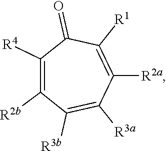

wherein R¹ is selected from hydrogen, halogen, —OH, —SH, —OC(O)Ar¹, —SC(O)Ar¹, —OC(O)(C1-C4 alkyl)Ar¹, —SC(O)(C1-C4 alkyl)Ar¹, and —OSO₂Ar¹; wherein Ar¹, when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a} and R^{2b} is independently selected from hydrogen, halogen, —OH, —CO₂H, and Ar²; wherein Ar², when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{3a} and R^{3b} is independently selected from hydrogen, halogen, C1-C6 alkyl, —C(O)R¹, —C(O)Ar³, and Ar³; wherein R¹¹, when present, is selected from C1-C4 alkyl and C3-C6 cycloalkyl; wherein Ar³, when present, is selected from C2-C5 heteroaryl and C6-C12 aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R⁴ is selected from hydrogen, halogen, and —OH, or a pharmaceutically acceptable salt thereof.

In various aspects, each of R^{2a}, R^{2b}, R^{3a}, R^{3b}, and R⁴ is hydrogen.

In various aspects, the troponoid has a structure represented by a formula:

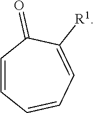

In various aspects, the troponoid has a structure represented by a formula:

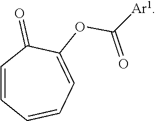

In various aspects, the troponoid is selected from:

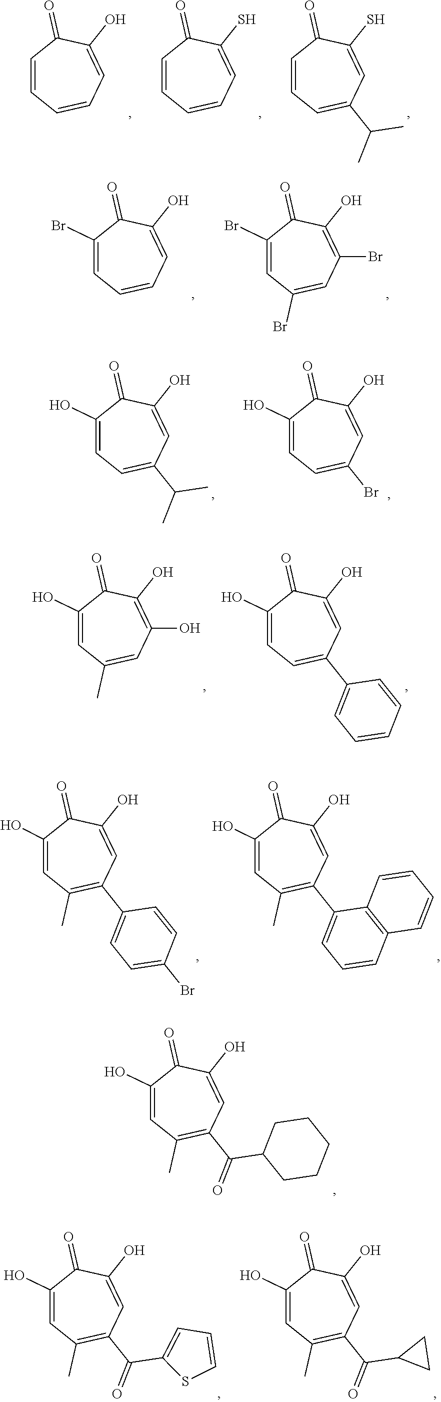

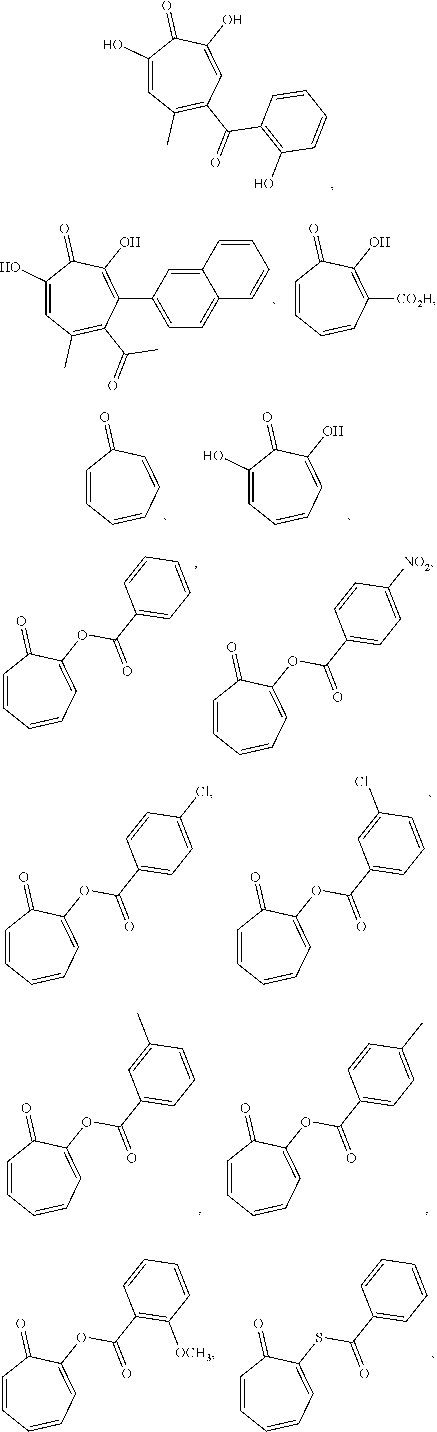

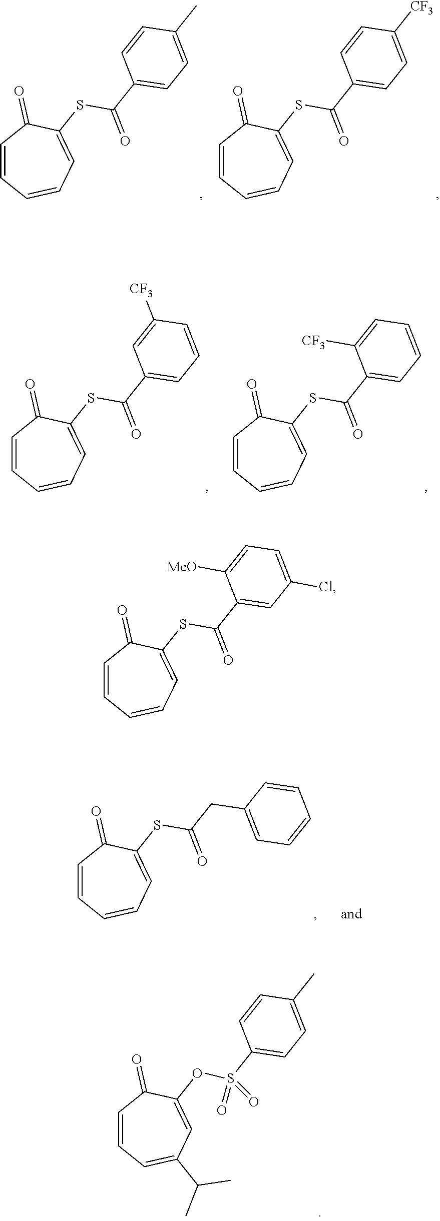

In various aspects, the troponoid is selected from:

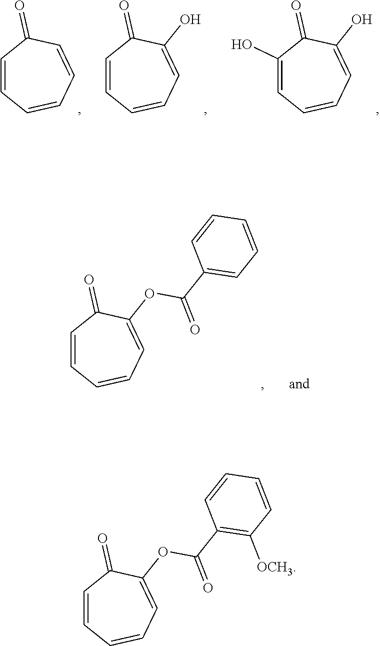

In various aspects, the troponoid is selected from:

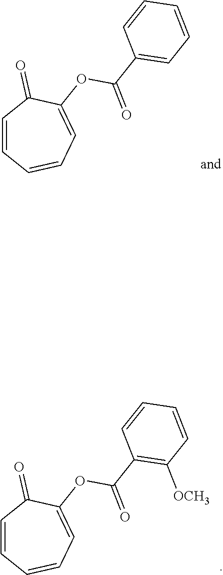 [SMILES: O=C(Oc1cccccc1=O)c1ccccc1 ; COc1ccccc1C(=O)Oc1cccccc1=O]

a. R¹

In one aspect, R¹ is selected from hydrogen, halogen, —OH, —SH, —OC(O)Ar¹, —SC(O)Ar¹, —OC(O)(C1-C4 alkyl)Ar¹, —SC(O)(C1-C4 alkyl)Ar¹, and —OSO₂Ar¹. In a further aspect, R¹ is selected from hydrogen, —F, -CL, —OH, —SH, —OC(O)Ar¹, —SC(O)Ar¹, —OC(O)CH₂Ar¹, —OC(O)CH₂CH₂Ar¹, —OC(O)CH₂CH₂CH₂Ar¹, —OC(O)CH(CH₃)CH₂Ar¹, —SC(O)CH₂Ar¹, —SC(O)CH₂CH₂Ar¹, —SC(O)CH₂CH₂CH₂Ar¹, —SC(O)CH(CH₃)CH₂Ar¹, and —OSO₂Ar¹. In a still further aspect, R¹ is selected from hydrogen, —F, -CL, —OH, —SH, —OC(O)Ar¹, —SC(O)Ar¹, —OC(O)CH₂Ar¹, —OC(O)CH₂CH₂Ar¹, —SC(O)CH₂Ar¹, —SC(O)CH₂CH₂Ar¹, and —OSO₂Ar¹. In yet a further aspect, R¹ is selected from hydrogen, —F, —Cl, —OH, —SH, —OC(O)Ar¹, —SC(O)Ar¹, —OC(O)CH₂Ar¹, —SC(O)CH₂Ar¹, and —OSO₂Ar¹.

In various aspects, R¹ is selected from hydrogen, halogen, —SH, —SC(O)Ar¹, —SC(O)(C1-C4 alkyl)Ar¹, and —OSO₂Ar¹. In a further aspect, R¹ is selected from hydrogen, —F, —Cl, —SH, —SC(O)Ar¹, —OC(O)CH(CH₃)CH₂Ar¹, —SC(O)CH₂Ar¹, —SC(O)CH₂CH₂Ar¹, —SC(O)CH₂CH₂CH₂Ar¹, —SC(O)CH(CH₃)CH₂Ar¹, and —OSO₂Ar¹. In a still further aspect, R¹ is selected from hydrogen, —F, —Cl, —SH, —SC(O)Ar¹, —SC(O)CH₂Ar¹, —SC(O)CH₂CH₂Ar¹, and —OSO₂Ar¹. In yet a further aspect, R¹ is selected from hydrogen, —F, —Cl, SH, —SC(O)Ar¹, —SC(O)CH₂Ar¹, and —OSO₂Ar¹.

In various aspects, R¹ is selected from hydrogen, halogen, —OH, —OC(O)Ar¹, and —OC(O)(C1-C4 alkyl)Ar¹. In a further aspect, R¹ is selected from hydrogen, —F, —Cl, —OH, —OC(O)Ar¹, —OC(O)CH₂Ar¹, —OC(O)CH₂CH₂Ar¹, —OC(O)CH₂CH₂CH₂Ar¹, and —OC(O)CH(CH₃)CH₂Ar¹. In a still further aspect, R¹ is selected from hydrogen, —F, —Cl, —OH, —OC(O)Ar¹, —OC(O)CH₂Ar¹, and —OC(O)CH₂CH₂Ar¹. In yet a further aspect, R¹ is selected from hydrogen, —F, —Cl, —OH, —OC(O)Ar¹, and —OC(O)CH₂Ar¹.

In various aspects, R¹ is selected from hydrogen, halogen, —OH, and —SH. In a further aspect, R¹ is selected from hydrogen, —F, —Cl, —OH, and —SH. In a still further aspect, R¹ is selected from hydrogen, —Cl, —OH, and —SH. In yet a further aspect, R¹ is selected from hydrogen, —F, —OH, and —SH.

In various aspects, R¹ is selected from hydrogen and halogen. In a further aspect, R¹ is selected from hydrogen, —F, —Cl, and —Br. In a still further aspect, R¹ is selected from hydrogen, —Cl, and —F. In yet a further aspect, R¹ is selected from hydrogen and —F.

In various aspects, R¹ is hydrogen.

In various aspects, R¹ is selected from hydrogen, —OH, and —OC(O)Ar¹.

In various aspects, R¹ is —OC(O)Ar¹.

b. R^{2a} and R^{2b}

In one aspect, each of R^{2a} and R^{2b} is independently selected from hydrogen, halogen, —OH, —CO₂H, and Ar². In a further aspect, each of R^{2a} and R^{2b} is independently selected from hydrogen, —F, —Cl, —OH, —CO₂H, and Ar². In a still further aspect, each of R^{2a} and R^{2b} is independently selected from hydrogen, —F, —OH, —CO₂H, and Ar².

In various aspects, each of R^{2a} and R^{2b} is independently selected from hydrogen and halogen. In a still further aspect, each of R^{2a} and R^{2b} is independently selected from hydrogen, —F, —Cl, and —Br. In yet a further aspect, each of R^{2a} and R^{2b} is independently selected from hydrogen, —F, and —Cl. In an even further aspect, each of R^{2a} and R^{2b} is independently selected from hydrogen and —F.

In various aspects, each of R^{2a} and R^{2b} is independently selected from hydrogen and —OH. In various aspects, each of R^{2a} and R^{2b} is independently selected from hydrogen and —CO₂H. In various aspects, each of R^{2a} and R^{2b} is independently selected from hydrogen and Ar².

In various aspects, each of R^{2a} and R^{2b} is hydrogen.

c. R^{3a} and R^{3b}

In one aspect, each of R^{3a} and R^{3b} is independently selected from hydrogen, halogen, C1-C6 alkyl, —C(O)R¹¹, —C(O)Ar³, and Ar³. In a further aspect, each of R^{3a} and R^{3b} is independently selected from hydrogen, —F, —Cl, methyl, ethyl, n-propyl, isopropyl, —C(O)R¹¹, —C(O)Ar³, and Ar³. In a still further aspect, each of R^{3a} and R^{3b} is independently selected from hydrogen, —F, —Cl, methyl, ethyl, —C(O)R¹¹, —C(O)Ar³, and Ar³. In yet a further aspect, each of R^{3a} and R^{3b} is independently selected from hydrogen, —F, —Cl, methyl, —C(O)R¹¹, —C(O)Ar³, and Ar³.

In various aspects, each of R^{3a} and R^{3b} is independently selected from hydrogen and C1-C6 alkyl. In a further aspect, each of R^{3a} and R^{3b} is independently selected from hydrogen, methyl, ethyl, n-propyl, and isopropyl. In a still further aspect, each of R^{3a} and R^{3b} is independently selected from hydrogen, methyl, and ethyl. In yet a further aspect, each of R^{3a} and R^{3b} is independently selected from hydrogen and methyl.

In various aspects, each of R^{3a} and R^{3b} is independently selected from hydrogen, —C(O)R¹¹, —C(O)Ar³, and Ar³. In a further aspect, each of R^{3a} and R^{3b} is independently selected from hydrogen, —C(O)Ar³, and Ar³. In a still further aspect, each of R^{3a} and R^{3b} is independently selected from hydrogen and —C(O)R¹. In yet a further aspect, each of R^{3a} and R^{3b} is independently selected from hydrogen and —C(O)Ar³. In an even further aspect, each of R^{3a} and R^{3b} is independently selected from hydrogen and Ar³.

In various aspects, each of R^{3a} and R^{3b} is independently selected from hydrogen and halogen. In a further aspect, each of R^{3a} and R^{3b} is independently selected from hydrogen, —F, —Cl, and —Br. In a still further aspect, each of R^{3a} and R^{3b} is independently selected from hydrogen, —F, and —Cl. In yet a further aspect, each of R^{3a} and R^{3b} is independently selected from hydrogen and —F.

In various aspects, each of R^{3a} and R^{3b} is hydrogen.

d. R⁴

In one aspect, R⁴ is selected from hydrogen, halogen, and —OH. In a further aspect, R⁴ is selected from hydrogen, —F, —Cl, and —OH. In a still further aspect, R⁴ is selected from hydrogen, —F, and —OH.

In various aspects, R⁴ is selected from hydrogen and halogen. In a further aspect, R⁴ is selected from hydrogen, —F, —Cl, and —Br. In a still further aspect, R⁴ is selected from hydrogen, —F, and —Cl. In yet a further aspect, R⁴ is selected from hydrogen and -Fl.

In various aspects, R⁴ is halogen. In a further aspect, R⁴ is selected from —F, —Cl, and —Br. In a still further aspect, R⁴ is selected from —F and —Cl. In yet a further aspect, R⁴ is -Fl. In an even further aspect, R⁴ is —Cl.

In various aspects, R⁴ is —OH.

In various aspects, R⁴ is hydrogen.

e. R¹¹

In one aspect, R¹¹, when present, is selected from C1-C4 alkyl and C3-C6 cycloalkyl. In a further aspect, R¹¹, when present, is selected from methyl, ethyl, n-propyl, isopropyl, cyclopropyl, cyclobutyl, and cyclopentyl. In a still further aspect, R¹¹, when present, is selected from methyl, ethyl, cyclopropyl, and cyclobutyl. In yet a further aspect, R¹¹, when present, is selected from methyl and cyclopropyl.

In various aspects, R¹¹, when present, is C1-C4 alkyl. In a further aspect, R¹¹, when present, is selected from methyl, ethyl, n-propyl, and isopropyl. In a still further aspect, R¹¹, when present, is selected from methyl and ethyl. In yet a further aspect, R¹¹, when present, is methyl.

In various aspects, R¹¹, when present, is C3-C6 cycloalkyl. In a further aspect, R¹¹, when present, is selected from cyclopropyl, cyclobutyl, and cyclopentyl. In a still further aspect, R¹¹, when present, is selected from cyclopropyl and cyclobutyl. In yet a further aspect, R¹¹, when present, is cyclopropyl.

In various aspects, R¹¹, when present, is selected from C1-C4 alkyl and C3-C6 cycloalkyl, and is unsubstituted.

f. AR¹

In one aspect, Ar¹, when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of C6-C12 aryls include, but are not limited to, phenyl, naphthyl, anthracenyl, naphthalenyl, and phenanthrenyl. In a further aspect, Ar¹, when present, is C6-C12 aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar¹, when present, is C6-C12 aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar¹, when present, is C6-C12 aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar¹, when present, is unsubstituted C6-C12 aryl.

In various aspects, Ar¹, when present, is C6 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar¹, when present, is C6 aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar¹, when present, is C6 aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar¹, when present, is C6 aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar¹, when present, is unsubstituted C6 aryl.

In various aspects, Ar¹, when present, is naphthyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar¹, when present, is naphthyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar¹, when present, is naphthyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar¹, when present, is naphthyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar¹, when present, is unsubstituted naphthyl.

g. AR²

In one aspect, Ar², when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of C6-C12 aryls include, but are not limited to, phenyl, naphthyl, anthracenyl, naphthalenyl, and phenanthrenyl. In a further aspect, Ar², when present, is C6-C12 aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar², when present, is C6-C12 aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar², when present, is C6-C12 aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar², when present, is unsubstituted C6-C12 aryl.

In various aspects, Ar², when present, is C6 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar², when present, is C6 aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar², when present, is C6 aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar², when present, is C6 aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar², when present, is unsubstituted C6 aryl.

In various aspects, Ar², when present, is naphthyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar², when present, is naphthyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar², when present, is naphthyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar², when present, is naphthyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar², when present, is unsubstituted naphthyl.

h. AR³

In one aspect, Ar³, when present, is selected from C2-C5 heteroaryl and C6-C12 aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar³, when present, is selected from C2-C5 heteroaryl and C6-C12 aryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is selected from C2-C5 heteroaryl and C6-C12 aryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar³, when present, is selected from C2-C5 heteroaryl and C6-C12 aryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is selected from C2-C5 heteroaryl and C6-C12 aryl, and is unsubstituted.

In various aspects, Ar³, when present, is C2-C5 heteroaryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of C2-C5 heterocycloalkyls include, but are not limited to, thiirane, oxirane, aziridine, thietane, azetidine, oxetane, pyrrolidine, imidazolidine, tetrahydrothiophene, tetrahydrofuran, piperidine, piperazine, thiane, and morpholine. In a further aspect, Ar³, when present, is C2-C5 heteroaryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is C2-C5 heteroaryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar³, when present, is C2-C5 heteroaryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is unsubstituted C2-C5 heteroaryl.

In various aspects, Ar³, when present, is thiophenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar³, when present, is thiophenyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is thiophenyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar³, when present, is thiophenyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is unsubstituted thiophenyl.

In various aspects, Ar³, when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of C6-C12 aryls include, but are not limited to, phenyl, naphthyl, anthracenyl, naphthalenyl, and phenanthrenyl. In a further aspect, Ar³, when present, is C6-C12 aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is C6-C12 aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar³, when present, is C6-C12 aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is unsubstituted C6-C12 aryl.

In various aspects, Ar³, when present, is C6 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar³, when present, is C6 aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is C6 aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar³, when present, is C6 aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is unsubstituted C6 aryl.

In various aspects, Ar³, when present, is naphthyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar³, when present, is naphthyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is naphthyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar³, when present, is naphthyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar³, when present, is unsubstituted naphthyl.

### D. NANOPARTICLES

In one aspect, disclosed are nanoparticles comprising a disclosed formulation. Thus, in various aspects, disclosed are nanoparticles comprising a liposome formulation comprising a troponoid and a lipid. In a further aspect, the troponoid has a structure represented by a formula:

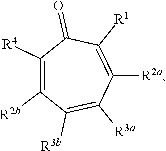

wherein R¹ is selected from hydrogen, halogen, —OH, —SH, —OC(O)Ar¹, —SC(O)Ar¹, —OC(O)(C1-C4 alkyl)Ar¹, —SC(O)(C1-C4 alkyl)Ar¹, and —OSO₂Ar¹; wherein Ar¹, when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a} and R^{2b} is independently selected from hydrogen, halogen, —OH, —CO₂H, and Ar²; wherein Ar², when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{3a} and R^{3b} is independently selected from hydrogen, halogen, C1-C6 alkyl, —C(O)R¹¹, —C(O)Ar³, and Ar³; wherein R¹¹, when present, is selected from C1-C4 alkyl and C3-C6 cycloalkyl; wherein Ar³, when present, is selected from C2-C5 heteroaryl and C6-C12 aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R⁴ is selected from hydrogen, halogen, and —OH, or a pharmaceutically acceptable salt thereof.

In one aspect, disclosed are nanoparticles comprising a liposome formulation comprising a troponoid, a lipid, and vitamin E.

In one aspect, disclosed are nanoparticles comprising a liposome formulation comprising: (a) a troponoid in an amount of about 15 wt % or less, wherein the troponoid is selected from:

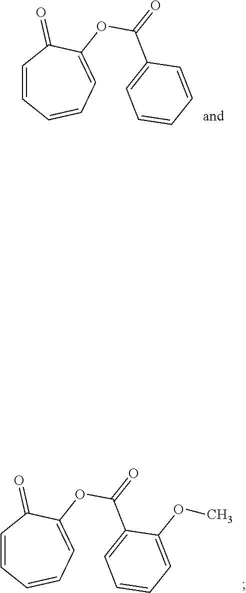 [SMILES: O=C(Oc1cccccc1=O)c1ccccc1 ; COc1ccccc1C(=O)Oc1cccccc1=O]

phosphatidylcholine in an amount of from about 60 wt % to about 99 wt %; cholesterol in an amount of about 20 wt % or less; and vitamin E in an amount of about 17 wt % or less.

### E. METHODS OF MAKING A FORMULATION

In one aspect, disclosed are methods of making a liposomal formulation, the method comprising: (a) providing a lipid solution comprising one or more lipids and an organic solvent; (b) removing the solvent, thereby forming a lipid cake; (c) mixing the lipid cake with a hydration media, thereby forming a hydrated solution; and (d) extruding the hydrated solution, thereby forming a liposomal formulation, wherein either the lipid solution or the hydration media comprises a troponoid.

In one aspect, disclosed are methods of preparing liposomal troponoid compounds and other antibiotics comprising: (a) preparing a lipid solution including phosphatidylcholine, cholesterol, vitamin-E and an organic solvent; (b) removing said solvent and forming a lipid cake; (c) hydrating said lipid cake with a hydration media, which may include a troponoid compound in a proper solvent (e.g., DMSO or water) or a solvent or aqueous media (e.g., H₂O or buffer); (d) mixing said lipid cake in a mixer and sonicate to form a homogenous hydrated solution (the homogenization may be enhanced by subjecting said lipid cake to one or more freeze/thaw cycles); (e) extruding said lipid cake to form liposomes which can be used directly or processed as shown in (f)-(h); (f) adding a cryo-protective agent to said liposomes and lyophilizing said liposomes to provide a powder; and/or (g) dialyzing against water to remove the polar solvent DMSO using Spectra/Por® Cellulose Ester (CE) dialysis membranes with molecular weight cut off; and wherein a troponoid compound is added to said lipid solution in step (a) as shown in Example 6 or to said hydration media used in step (c) as shown in Examples 1-5.

In one aspect, disclosed are methods to fabricate a liposome with a disclosed formulation. The method of fabrication may include, for example, thin film hydration and extrusion using a liposome extruder. Other means of fabrication method include, but are not limited to, high pressure homogenization, reverse phase evaporation, and ethanol injection.

The method of formulating the liposomal hydrophobic troponoid compound formulation, generally includes adding a solvent containing troponoid compounds to the lipids (i.e., the phosphatidylcholine, cholesterol, and vitamin-E) to prepare a solution. The solvents preferably include chloroform, methanol, or a combination thereof at a 1:1 ratio. The solvent is preferably removed to form a lipid cake. The solvents may be removed using an evaporation system such as a nitrogen blow down dry evaporator or a rotary evaporator, or even a spray dryer. The lipid cake may then be further dried by, e.g., vacuum drying. Aqueous media, such as water, or a phosphate buffered solution or aqueous saline solution, is then added to the cake to hydrate the cake to form a homogenous lipid-drug solution.

Alternatively, the troponoid compounds may be dissolved in DMSO and they may be added together with the aqueous media. If the troponoid compounds are hydrophilic, they may be directly dissolved in the above aqueous media and directly used to hydrate the lipid cake. The hydrated cake may be mixed in a vortex mixer, sonication, and exposed to one or more freeze/thaw cycles (such as in the range of 3 to 10 cycles). Freezing may be facilitated by, e.g., liquid N2 and thawing may be facilitated by, e.g., a 40° C. sonicator bath. The thawed solution is preferably then extruded one or more times (such as in the range of 1 to 11) through one or more membranes to form liposomes. The extruder used may be, e.g., a mini-extruder, available from Avanti Polar lipids, a LIPEX extruder, available from Transferra, or even using a high-pressure homogenizer such as emulsiflex c5, available from Avestin. In preferred embodiments, two types membranes are used, a 0.2 μm (e.g., WHATMAN® polycarbonate track-etch) membrane and a 0.1 μm membrane. A cryo-protecting agent, such as sucrose is then preferably added to the liposomes and the liposomes may be flash frozen using liquid nitrogen and is then lyophilized into a liposomal drug powder. The liposomal powder may then be rehydrated with an aqueous media to form a reconstituted solution of the liposomal troponoid compounds.

In various aspects, the liposomal troponoid compounds may be shipped either as powder, as a reconstituted solution, or combined with secondary topical treatments such as silicone gel (e.g., KELO-COTE 10 available from Senvie Skin Care), 2-(2-ethoxyethoxy)ethanol (e.g. TRANSCUTOL available from Gattefossé) or glyceryl caprylate/caprate (e.g., CAPMUL MCM EP available from ABITEC corporation).

In various aspects, said final dried topical liposome formulation (without any solvent or water, or cyroprotective agent) comprises said phosphatidylcholine present in the range of 60 wt % to 99 wt %, cholesterol present in the range of 0 wt % to 20 wt %, vitamin-E present in the range of 0 wt % to 17 wt %, said troponoid compound present in the range of 0 wt % to 15 wt %, wherein said formulation is selected to equal 100 wt %.

In various aspects, the lipid composition may be further modified for intravenous application by adding a stealth lipid such as 1,2-distearoyl-sn-glycero-3-phosphoethanolamine-N-[amino(polyethylene glycol)-2000 (DSPE-PEG), or mPEG-DSPE (CAS No. 474922-77-5).

In various aspects, the PC can be either from soy or egg York sources. For example, Lipoid S-100 from soy source may be used.

In various aspects, the troponoid compound includes compound 62, compound 285, or many other troponoid compound derivatives.

In various aspects, the drug may also be other antibiotics such as gentamicin or Ceftriaxone, or other antibacterial and antifungal agents.

In various aspects, said cryo-protective agent comprises sucrose, or trehalose, or other cryoprotective compounds. After reconstitution in an aqueous media, the cryo-protective agent can be in the range of 0-15 w/v %.

In various aspects, the troponoid has a structure represented by a formula:

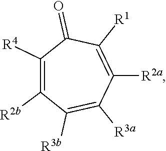

wherein R¹ is selected from hydrogen, halogen, —OH, —SH, —OC(O)Ar¹, —SC(O)Ar¹, —OC(O)(C1-C4 alkyl)Ar¹, —SC(O)(C1-C4 alkyl)Ar¹, and —OSO₂Ar¹; wherein Ar¹, when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a} and R^{2b} is independently selected from hydrogen, halogen, —OH, —CO₂H, and Ar²; wherein Ar², when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{3a} and R^{3b} is independently selected from hydrogen, halogen, C1-C6 alkyl, —C(O)R¹, —C(O)Ar³, and Ar³; wherein R¹¹, when present, is selected from C1-C4 alkyl and C3-C6 cycloalkyl; wherein Ar³, when present, is selected from C2-C5 heteroaryl and C6-C12 aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R⁴ is selected from hydrogen, halogen, and —OH, or a pharmaceutically acceptable salt thereof.

In various aspects, the troponoid is selected from:

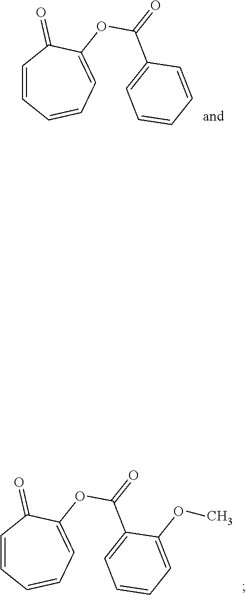 [SMILES: O=C(Oc1cccccc1=O)c1ccccc1 ; COc1ccccc1C(=O)Oc1cccccc1=O]

In various aspects, the troponoid is present in an amount of about 15 wt % or less, based on the weight of the formulation. Thus, in various further aspects, the troponoid is present in an amount of about 30 wt % or less, 25 wt % or less, 20 wt % or less, 15 wt % or less, 10 wt % or less, 5 wt % or less, or 1 wt % or less, based on the weight of the formulation.

In various aspects, the lipid solution comprises a lipid and vitamin E. In a further aspect, the lipid solution comprises a lipid, a troponoid, and vitamin E.

In various aspects, the lipid solution comprises one or more of phosphatidylcholine, cholesterol, and vitamin E. In a further aspect, the lipid solution comprises phosphatidylcholine, cholesterol, and vitamin E.

In various aspects, the lipid solution comprises the troponoid. In a further aspect, the hydration media comprises the troponoid.

In various aspects, the lipid is present in an amount of from about 60 w % to about 99 wt %, based on the weight of the formulation. Thus, in various aspects, the lipid is present in an amount of from about 50 wt % to about 99 wt %, 60 wt % to about 99 wt %, 70 wt % to about 99 wt %, 80 wt % to about 99 wt %, 90 wt % to about 99 wt %, 60 wt % to about 90 wt %, 60 wt % to about 80 wt %, or 60 wt % to about 70 wt %, based on the weight of the formulation.

In various aspects, the lipid is sourced from soybeans or egg yolk. In a further aspect, the lipid is sourced from soybeans. In a still further aspect, the lipid is sourced from egg yolk.

In various aspects, the method further comprises adding cholesterol to the formulation. The cholesterol can be present in an amount of about 30 wt % or less, 25 wt % or less, 20 wt % or less, 15 wt % or less, 10 w % or less, or 5 wt % or less, based on the weight of the formulation.

In various aspects, the method further comprises adding vitamin E to the formulation. The vitamin E can be present in an amount of about 17 wt % or less, based on the weight of the formulation. Thus, in various further aspects, the vitamin E can be present in an amount of about 25 wt % or less, 23 wt % or less, 20 wt % or less, 17 wt % or less, 15 wt % or less, 12 wt % or less, 12 wt % or less, 10 wt % or less, 8 wt % or less, 6 wt % or less, 5 wt % or less, 4 wt % or less, 3 wt % or less, 2 wt % or less, or 1 wt % or less, based on the weight of the formulation.

In various aspects, the hydrated solution is homogenous.

In various aspects, the method further comprises subjecting the lipid cake to one or more freeze/thaw cycles.

In various aspects, the method further comprises adding a cryo-protective agent to the liposome formulation. Examples of cryo-protective agents include, but are not limited to, sucrose and trehalose. The cryo-protective agent can be present in an amount of about 15 wt % or less, based on the weight of the formulation. Thus, in various further aspects, the cryo-protective agent can be present in an amount of about 25 wt % or less, 20 wt % or less, 15 wt % or less, 10 wt % or less, or 5 wt % or less, based on the weight of the formulation. In a still further aspect, the cryo-protective agent can be present in an amount of from about 5 wt % to about 25 wt %, from about 5 wt % to about 20 wt %, from about 5 wt % to about 15 wt %, from about 5 wt % to about 10 wt %, from about 10 wt % to about 25 wt %, from about 15 wt % to about 25 wt %, from about 20 wt % to about 25 wt %, or from about 5 wt % to about 15 wt %, based on the weight of the formulation.

In various aspects, the method further comprises dialyzing against water.

In various aspects, the method further comprises adding a stealth lipid to the formulation. Examples of stealth lipids include, but are not limited to, 1,2-distearoyl-sn-glycero-3-phosphoethanolamine-N-[amino(polyethylene glycol) (DSPE-PEG), mPEG-DSPE, and 1,2-dioleoyl-sn-glycero-3-phosphoethanolamine-N-[amino(polyethylene glycol)](DOPE), or pharmaceutically acceptable salts thereof.

In various aspects, the method further comprises adding a positively charged molecule or a positively charged lipid to the formulation. Examples of positively charged molecules include, but are not limited to, stearyl amines. Examples of positively charged lipids include, but are not limited to, 1,2-dimyristoyl-3-trimethylammonium-propane, 1,2-dilauroyl-sn-glycero-3-ethylphosphocholine, and pharmaceutically acceptable salts thereof.

In various aspects, the method further comprises adding one or more of an antibiotic agent, an antibacterial agent, an antifungal agent, an antiviral agent, a chemotherapeutic agent, an anti-inflammatory agent, and a cardiac agent to the formulation.

Examples of antibiotic agents include, but are not limited to, lipopeptides, fluoroquinolone, lipoglycopeptides, macrolides, β-lactams such as penicillins, cephalosporins, monobactams, and carbapenems, lincosamides, streptogramins, aminoglycosides, quinolones, sulfonamides, tetracyclines, chloramphenicol, metronidazole, tinidazole, nitrofurantoin, glycopeptides, lipoglycopeptides, oxazolidinones, rifamycins, polypeptides, and tuberactinomycins. In a further aspect, the antibiotic agent is selected from gentamicin and ceftriaxone.

Examples of antibacterial agents include, but are not limited to, amoxicillin, ampicillin, azithromycin, aztreonam, azlocillin, bacitracin, carbenicillin, cefaclor, cefadroxil, cefamandole, cefazolin, cephalexin, cefdinir, cefditorin, cefepime, cefixime, cefoperazone, cefotaxime, cefoxitin, cefpodoxime, cefprozil, ceftazidime, ceftibuten, ceftizoxime, ceftriaxone, cefuroxime, chloramphenicol, cilastin, ciprofloxacin, clarithromycin, clavulanic acid, clinafloxacin, clindamycin, clofazimine, cloxacillin, colistin, dalbavancin, dalfopristin, demeclocycline, dicloxacillin, dirithromycin, doxycycline, erythromycin, enrofloxacin, enoxacin, enviomycin, ertepenem, ethambutol, flucloxacillin, fosfomycin, furazolidone, gatifloxacin, gentamicin, imipenem, isoniazid, kanamycin, linezolid, lomefloxacin, loracarbef, mafenide, moxifloxacin, meropenem, metronidazole, mezlocillin, minocycline, mupirocin, nafcillin, nalidixic acid, neomycin, netilmicin, nitrofurantoin, norfloxacin, ofloxacin, oritavancin, oxytetracycline, penicillin, piperacillin, platensimycin, polymixin B, quinupristin, retapamulin, rifabutin, rifampin, rifapentine, roxithromycin, sparfloxacin, spectinomycin, sulbactam, sulfacetamide, sulfamethizole, sulfamethoxazole, teicoplanin, telithromycin, telavancin, temafloxacin, tetracycline, thioacetazone, thioridazine, ticarcillin, tinidazole, tobramycin, torezolid, tosufloxacin, trimethoprim, troleandomycin, trovafloxacin, and vancomycin, or combinations thereof.

Examples of antifungal agents include, but are not limited to, clotrimazole, econazole, miconazole, terbinafine, fluconazole, ketoconazole, and amphotericin.

In a further aspect, the formulation is formulated as a as a liquid, a lyophilized powder, a cream, or an ointment.

In a further aspect, the formulation is substantially free of dimethylsulfoxide (DMSO), methanol, and/or chloroform.

In a further aspect, the formulation is substantially free of organic solvent. Examples of organic solvents include, but are not limited to, methylene chloride, 1,1,1-trichloroethane, carbon tetrachloride, trichloroethylene, ethanol, n-hexane, methyl ethyl ketone, formaldehyde, acetone, benzene, toluene, and mixtures thereof.

### F. METHODS OF USING THE FORMULATIONS

The formulations of the invention are useful in treating or controlling disorders associated with a viral infection, an antimicrobial infection, cancer, an inflammatory disease, or a cardiovascular disease. To treat or control the disorder, the formulations are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a viral infection, an antimicrobial infection, cancer, an inflammatory disease, or a cardiovascular disease.

The formulations can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a viral infection, an antimicrobial infection, cancer, an inflammatory disease, or a cardiovascular disease.

The therapeutically effective amount or dosage of the formulation (or of the compound within the formulation) can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

1. Treatment Methods

The formulations disclosed herein are useful for treating or controlling disorders associated with a viral infection, an antimicrobial infection, cancer, an inflammatory disease, or a cardiovascular disease. Thus, provided is a method comprising administering an effective amount of a disclosed formulation to a subject. In a further aspect, the method can be a method for treating a viral infection, an antimicrobial infection, cancer, an inflammatory disease, or a cardiovascular disease.

The liposomal troponoid(s) may be applied topically onto a subject, and preferably onto a wound. Thus, in various aspects, disclosed are methods of applying a liposomal troponoid compound formulation, comprising the liposome (either reconstituted powder or liquid form or added to a spray device or a cream/ointment), topically to a subject (e.g., human or animals). Other routes of application includes intranasal, intravaginal, oral, intramuscular, intradermal, or intravenous administration. Topical application of the formulation may be understood as the application of the liposomes (powdered, reconstituted or incorporated into a secondary topical treatment) onto the skin or mucosal membranes of the subject. Wounds may be understood as an injury to tissue, including but not limited to the skin or mucosal membranes, wherein the injury may include the damage or breach of the tissue. A subject may be understood as a human or other mammal, such as rabbits, mice, dogs, etc.

The liposomal formulations were found to kill drug-resistant bacteria such as Sa #10, which is mupirocin-resistant MDRSA. In other words, traditional mupirocin ointment cannot kill this type of bacteria; however, the disclosed formulated troponoids such as, for example, compound 285 and 62, can kill this type of bacteria.

a. Treating a Disease or Disorder

In one aspect, disclosed are methods for treating a disease or disorder in a subject, the method comprising administering to the subject an effective amount of a disclosed formulation, wherein the disease or disorder is a viral infection, an antimicrobial infection, cancer, an inflammatory disease, or a cardiovascular disease.

In one aspect, disclosed are methods for treating a disease or disorder in a subject, the method comprising administering to the subject an effective amount of a liposome formulation comprising a troponoid and a lipid, wherein the disease or disorder is a viral infection, an antimicrobial infection, cancer, an inflammatory disease, or a cardiovascular disease.

In one aspect, disclosed are methods for treating a disease or disorder in a subject, the method comprising administering to the subject an effective amount of a liposome formulation comprising a troponoid having a structure represented by a formula:

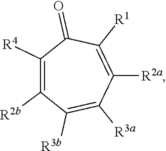

wherein R¹ is selected from hydrogen, halogen, —OH, —SH, —OC(O)Ar¹, —SC(O)Ar¹, —OC(O)(C1-C4 alkyl)Ar¹, —SC(O)(C1-C4 alkyl)Ar¹, and —OSO₂Ar¹; wherein Ar¹, when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a} and R^{2b} is independently selected from hydrogen, halogen, —OH, —CO₂H, and Ar²; wherein Ar², when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{3a} and R^{3b} is independently selected from hydrogen, halogen, C1-C6 alkyl, —C(O)R¹¹, —C(O)Ar³, and Ar³; wherein R¹¹, when present, is selected from C1-C4 alkyl and C3-C6 cycloalkyl; wherein Ar³, when present, is selected from C2-C5 heteroaryl and C6-C12 aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R⁴ is selected from hydrogen, halogen, and —OH, or a pharmaceutically acceptable salt thereof, wherein the disease or disorder is a viral infection, an antimicrobial infection, cancer, an inflammatory disease, or a cardiovascular disease.

In one aspect, disclosed are methods for treating a disease or disorder in a subject, the method comprising administering to the subject an effective amount of a liposome formulation comprising a troponoid, a lipid, and vitamin E, wherein the disease or disorder is a viral infection, an antimicrobial infection, cancer, an inflammatory disease, or a cardiovascular disease.

In one aspect, disclosed are methods for treating a disease or disorder in a subject, the method comprising administering to the subject an effective amount of a liposome formulation comprising: (a) a troponoid in an amount of about 15 wt % or less, wherein the troponoid is selected from:

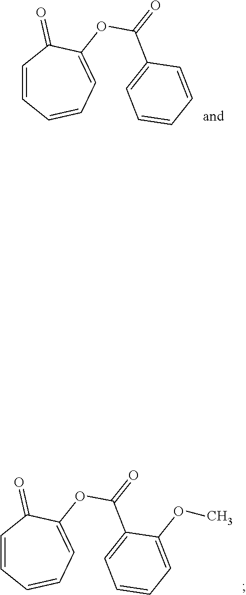 [SMILES: O=C(Oc1cccccc1=O)c1ccccc1 ; COc1ccccc1C(=O)Oc1cccccc1=O]

phosphatidylcholine in an amount of from about 60 wt % to about 99 wt %; cholesterol in an amount of about 20 wt % or less; and vitamin E in an amount of about 17 wt % or less, wherein the disease or disorder is a viral infection, an antimicrobial infection, cancer, an inflammatory disease, or a cardiovascular disease.

In a further aspect, the subject has been diagnosed with a need for treatment of the disease or disorder prior to the administering step.

In a further aspect, the subject is a mammal. In a still further aspect, the mammal is a human.

In a further aspect, the method further comprises the step of identifying a subject in need of treatment of the disease or disorder.

In a further aspect, administering is topical, oral, intranasal, intramuscular, or subcutaneous administration. In a still further aspect, administering is topical administration.

In a further aspect, the effective amount is a therapeutically effective amount. In a still further aspect, the effective amount is a prophylactically effective amount.

In a further aspect, the disease or disorder is an antimicrobial infection.

Examples of antimicrobial infections include, but are not limited to, bacterial infections and fungal infections.

In a further aspect, the antimicrobial infection is a bacterial infection.

Examples of bacterial infections include, but are not limited to, M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neisseria meningitidis, Neisseria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species. In a still further aspect, the bacterial infection is Staphylococcus aureus.

In a further aspect, the antimicrobial infection is a fungal infection. Examples of fungal infections include, but are not limited to, ringworm, a Candida infection, a fungal nail infection, Blastomyces infection, Cryptococcus gattii infection, Paracoccidioides infection, Coccidioides infection, and Histoplasmosis infection.

In a further aspect, the disease or disorder is a viral infection. Examples of viral infections include, but are not limited to, human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), human coronavirus, and zika (ZIKV).

In a further aspect, the disease or disorder is cancer. Examples of cancers include, but are not limited to, a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma).

In a further aspect, the disease or disorder is a cardiovascular disease. Examples of cardiovascular diseases include, but are not limited to, heart failure and myocardial infarction.

In a further aspect, the method further comprises administering to the subject an effective amount of at least one agent selected from an antibiotic agent, an antibacterial agent, an antifungal agent, an antiviral agent, a chemotherapeutic agent, an anti-inflammatory agent, and a cardiac agent.

Examples of antiviral agents include, but are not limited to, acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscamet sodium, fosfonet sodium, FTC, ganciclovir, ganciclovir sodium, GSK 1265744, 9-2-hydroxy-ethoxy methylguanine, ibalizumab, idoxuridine, interferon, 5-iodo-2′-deoxyuridine, IQP-0528, kethoxal, lamivudine, lobucavir, maraviroc, memotine pirodavir, penciclovir, raltegravir, ribavirin, rimantadine hydrochloride, rilpivirine (TMC-278), saquinavir mesylate, SCH-C, SCH-D, somantadine hydrochloride, sorivudine, statolon, stavudine, T20, tilorone hydrochloride, TMC120, TMC125, trifluridine, trifluorothymidine, tenofovir, tenofovir alefenamide, tenofovir disoproxyl fumarate, prodrugs of tenofovir, UC-781, UK-427, UK-857, valacyclovir, valacyclovir hydrochloride, vidarabine, vidarabine phosphate, vidarabine sodium phosphate, viroxime, zalcitabene, zidovudine, and zinviroxime.

Examples of chemotherapeutic agents include, but are not limited to, an alkylating agent (e.g., carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, streptozocin), an antimetabolite agent (e.g., gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, thioguanine), an antineoplastic antibiotic agent (e.g., doxorubicin, mitoxantrone, bleomycin, daunorubicin, dactinomycin, epirubicin, idarubicin, plicamycin, mitomycin, pentostatin, valrubicin), a mitotic inhibitor agent (e.g., irinotecan, topotecan, rubitecan, cabazitaxel, docetaxel, paclitaxel, etopside, vincristine, ixabepilone, vinorelbine, vinblastine, teniposide), and a mTor inhibitor agent (e.g., everolimus, siroliumus, temsirolimus).

Examples of anti-inflammatory agents include, but are not limited to, non-steroidal anti-inflammatory drug(s) (NSAIDs), cytokine suppressive anti-inflammatory drug(s) (CSAIDs), MK-966 (COX-2 Inhibitor), iloprost, methotrexate, thalidomide and thalidomide-related drugs (e.g., CELGEN), leflunomide, tranexamic acid, T-614, prostaglandin E1, tenidap, naproxen, meloxicam, ibuprofen, piroxicam, diclofenac, indomethacin, sulfasalazine, azathioprine, ICE inhibitors (inhibitors of the enzyme interleukin-1l converting enzyme), zap-70 and/or lck inhibitors (inhibitor of the tyrosine kinase zap-70 or lck), corticosteroid anti-inflammatory drugs (e.g., SB203580), TNF-convertase inhibitors, interleukin-17 inhibitors, gold, penicillamine, chloroquine, hydroxychloroquine, chlorambucil, cyclophosphamide, cyclosporine, total lymphoid irradiation, anti-thymocyte globulin, CD5-toxins, lobenzarit disodium, cytokine regulating agents (CRAs) HP228 and HP466, prednisone, orgotein, glycosaminoglycan polysulfate, minocycline, anti-IL2R antibodies, auranofin, phenylbutazone, meclofenamic acid, flufenamic acid, intravenous immune globulin, zileuton, mycophenolic acid (RS-61443), tacrolimus (FK-506), sirolimus (rapamycin), amiprilose (therafectin), cladribine (2-chlorodeoxyadenosine), azaribine, and methotrexate.

Examples of cardiac agents include, but are not limited to, quinapril, perindopril, nifedipine, ramipril, hydralazine, aspirin, ticagrelor, captopril, carvedilol, losartan, isosorbide dinitrate, valsartan, prasugrel, isosorbide mononitrate, eplerenone, and isosorbide dinitrate.

In a further aspect, the formulation and the agent are administered sequentially. In a still further aspect, the formulation and the agent are administered simultaneously.

In a further aspect, the formulation and the agent are co-packaged.

2. Use of Compounds

In one aspect, the invention relates to the use of a disclosed formulation. In a further aspect, a use relates to the manufacture of a medicament for the treatment of a viral infection, an antimicrobial infection, cancer, an inflammatory disease, and/or a cardiovascular disease in a subject.

In a further aspect, the use relates to a process for preparing a disclosed formulation for use as a medicament.

In a further aspect, the use related to a method of preparing liposomal formulations containing atroponoid compound, as further described here. For example, in various aspects, the method includes preparing a lipid solution including phosphatidylcholine, cholesterol, vitamin-E, and an organic solvent, removing the solvent, and forming a lipid cake. The lipid cake may then be hydrated with a first aqueous media. The hydrated cake may be mixed in a mixer, subjected to one or more freeze/thaw cycles and then extruded to form liposomes. A cryo-protective agent may be added to the liposomes and the liposomes may be lyophilized to provide a powder. A hydrophobic troponoid compound is added to the organic solvent lipid solution or to the first aqueous media used for hydrating the lipid cake.

In a further aspect, the use relates to a method of applying a liposomal formulation containing a troponoid compound, as further described herein. The method includes providing a topical formulation of liposomal troponoid compounds as a powder or a liquid, or added to a cream and ointment. The liposomal formulation may also be subjected to other routes of administration such as oral, intranasal, intramuscular, subcutaneous, etc. For the intravenous application, the lipid composition can be further modified by adding mPEG-DSPE or other stealth lipids in the amount of up to 15 wt % in the lipid composition.

In one aspect, the use is characterized in that the subject is a human.

In various aspects, the use relates to a treatment of an antimicrobial infection. In one aspect, the use is characterized in that the antimicrobial infection is a bacterial infection or a fungal infection. In one aspect, the use is characterized in that the bacterial infection is M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neisseria meningitidis, Neisseria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, or other Yersinia species. In one aspect, the use is characterized in that the bacterial infection is Staphylococcus aureus. In one aspect, the use is characterized in that the fungal infection ringworm, a Candida infection, a fungal nail infection, Blastomyces infection, Cryptococcus gattii infection, Paracoccidioides infection, Coccidioides infection, or Histoplasmosis infection.

In various aspects, the use relates to a treatment of a viral infection in a subject. In one aspect, the use is characterized in that the viral infection is human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), human coronavirus, or zika (ZIKV).

In various aspects, the use relates to a treatment of cancer in a subject. In one aspect, the use is characterized in that the cancer is a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, or plasma cell neoplasm (myeloma).

In various aspects, the use relates to a treatment of an inflammatory disease in a subject. In one aspect, the use is characterized in that the inflammatory disease is rheumatoid arthritis, insulin-dependent diabetes mellitus, multiple sclerosis, myasthenia gravis, Crohn's disease, autoimmune nephritis, primary biliary cirrhosis, psoriasis, acute pancreatitis, allograph rejection, allergic inflammation, inflammatory bowel disease, septic shock, osteoporosis, osteoarthritis, or cognitive deficits induced by neuronal inflammation.

In various aspects, the use relates to a treatment of a cardiovascular disease in a subject. In one aspect, the use is characterized in that the cardiovascular disease is heart failure or myocardial infarction.

In a further aspect, the use relates to the manufacture of a medicament for the treatment of a viral infection in a subject.

In a further aspect, the use relates to antagonism or inhibition of a viral infection in a subject. In a further aspect, the use relates to modulating viral activity in a subject. In a still further aspect, the use relates to modulating viral activity in a cell. In yet a further aspect, the subject is a human.

It is understood that the disclosed uses can be employed in connection with the disclosed compounds, formulations, products of disclosed methods of making, methods, and kits. In a further aspect, the invention relates to the use of a disclosed formulation or a disclosed product in the manufacture of a medicament for the treatment of a disease or disorder in a mammal. In a further aspect, the disease or disorder is selected from a viral infection, an antimicrobial infection, cancer, an inflammatory disease, or a cardiovascular disease.

3. Manufacture of a Medicament

In one aspect, the invention relates to a method for the manufacture of a medicament for treating a disease or disorder in a subject in need thereof, the method comprising combining a formulation comprising a therapeutically effective amount of a disclosed compound, or product of a disclosed method, with a pharmaceutically acceptable carrier or diluent, wherein the disease or disorder is a viral infection, an antimicrobial infection, cancer, an inflammatory disease, or a cardiovascular disease.

As regards these applications, the present method includes the administration to an animal, particularly a mammal, and more particularly a human, of a formulation comprising a therapeutically effective amount of the compound effective in treating the disease or disorder. The dose administered to an animal, particularly a human, in the context of the present invention should be sufficient to affect a therapeutic response in the animal over a reasonable time-frame. One skilled in the art will recognize that dosage will depend upon a variety of factors including the condition of the animal and the body weight of the animal.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound, formulation, or a product of a disclosed method of making, or a pharmaceutically acceptable salt, solvate, or polymorph thereof, with a pharmaceutically acceptable carrier or diluent.

4. Kits

In one aspect, disclosed are kits comprising a disclosed formulation, and one or more of: (a) an agent selected from antibiotic agent, an antibiotic agent, an antibacterial agent, an antifungal agent, an antiviral agent, a chemotherapeutic agent, an anti-inflammatory agent, and a cardiac agent; and (b) instructions for treating a viral infection, an antimicrobial infection, cancer, an inflammatory disease, and/or a cardiovascular disease.

In one aspect, disclosed are kits comprising a liposome formulation comprising a troponoid and a lipid, and one or more of: (a) an agent selected from antibiotic agent, an antibiotic agent, an antibacterial agent, an antifungal agent, an antiviral agent, a chemotherapeutic agent, an anti-inflammatory agent, and a cardiac agent; and (b) instructions for treating a viral infection, an antimicrobial infection, cancer, an inflammatory disease, and/or a cardiovascular disease.

In one aspect, disclosed are kits comprising a liposome formulation comprising a troponoid having a structure represented by a formula:

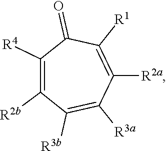

wherein R¹ is selected from hydrogen, halogen, —OH, —SH, —OC(O)Ar¹, —SC(O)Ar¹, —OC(O)(C1-C4 alkyl)Ar¹, —SC(O)(C1-C4 alkyl)Ar¹, and —OSO₂Ar¹; wherein Ar¹, when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a} and R^{2b} is independently selected from hydrogen, halogen, —OH, —CO₂H, and Ar²; wherein Ar², when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{3a} and R^{3b} is independently selected from hydrogen, halogen, C1-C6 alkyl, —C(O)R¹¹, —C(O)Ar³, and Ar³; wherein R¹¹, when present, is selected from C1-C4 alkyl and C3-C6 cycloalkyl; wherein Ar³, when present, is selected from C2-C5 heteroaryl and C6-C12 aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein R⁴ is selected from hydrogen, halogen, and —OH, or a pharmaceutically acceptable salt thereof, and one or more of (a) an agent selected from antibiotic agent, an antibiotic agent, an antibacterial agent, an antifungal agent, an antiviral agent, a chemotherapeutic agent, an anti-inflammatory agent, and a cardiac agent; and (b) instructions for treating a viral infection, an antimicrobial infection, cancer, an inflammatory disease, and/or a cardiovascular disease.

In one aspect, disclosed are kits comprising a liposome formulation comprising a troponoid, a lipid, and vitamin E, and one or more of: (a) an agent selected from antibiotic agent, an antibiotic agent, an antibacterial agent, an antifungal agent, an antiviral agent, a chemotherapeutic agent, an anti-inflammatory agent, and a cardiac agent; and (b) instructions for treating a viral infection, an antimicrobial infection, cancer, an inflammatory disease, and/or a cardiovascular disease.

In one aspect, disclosed are kits comprising a liposome formulation comprising: (a) a troponoid in an amount of about 15 wt % or less, wherein the troponoid is selected from:

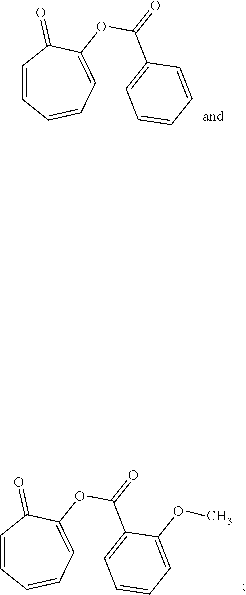 [SMILES: O=C(Oc1cccccc1=O)c1ccccc1 ; COc1ccccc1C(=O)Oc1cccccc1=O]

phosphatidylcholine in an amount of from about 60 wt % to about 99 wt %; cholesterol in an amount of about 20 wt % or less; and vitamin E in an amount of about 17 wt % or less, and one or more of (a) an agent selected from antibiotic agent, an antibiotic agent, an antibacterial agent, an antifungal agent, an antiviral agent, a chemotherapeutic agent, an anti-inflammatory agent, and a cardiac agent; and (b) instructions for treating a viral infection, an antimicrobial infection, cancer, an inflammatory disease, and/or a cardiovascular disease.

Examples of antiviral agents include, but are not limited to, acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscamet sodium, fosfonet sodium, FTC, ganciclovir, ganciclovir sodium, GSK 1265744, 9-2-hydroxy-ethoxy methylguanine, ibalizumab, idoxuridine, interferon, 5-iodo-2′-deoxyuridine, IQP-0528, kethoxal, lamivudine, lobucavir, maraviroc, memotine pirodavir, penciclovir, raltegravir, ribavirin, rimantadine hydrochloride, rilpivirine (TMC-278), saquinavir mesylate, SCH-C, SCH-D, somantadine hydrochloride, sorivudine, statolon, stavudine, T20, tilorone hydrochloride, TMC120, TMC125, trifluridine, trifluorothymidine, tenofovir, tenofovir alefenamide, tenofovir disoproxyl fumarate, prodrugs of tenofovir, UC-781, UK-427, UK-857, valacyclovir, valacyclovir hydrochloride, vidarabine, vidarabine phosphate, vidarabine sodium phosphate, viroxime, zalcitabene, zidovudine, and zinviroxime.

Examples of chemotherapeutic agents include, but are not limited to, an alkylating agent (e.g., carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, streptozocin), an antimetabolite agent (e.g., gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, thioguanine), an antineoplastic antibiotic agent (e.g., doxorubicin, mitoxantrone, bleomycin, daunorubicin, dactinomycin, epirubicin, idarubicin, plicamycin, mitomycin, pentostatin, valrubicin), a mitotic inhibitor agent (e.g., irinotecan, topotecan, rubitecan, cabazitaxel, docetaxel, paclitaxel, etopside, vincristine, ixabepilone, vinorelbine, vinblastine, teniposide), and a mTor inhibitor agent (e.g., everolimus, siroliumus, temsirolimus).

Examples of anti-inflammatory agents include, but are not limited to, non-steroidal anti-inflammatory drug(s) (NSAIDs), cytokine suppressive anti-inflammatory drug(s) (CSAIDs), MK-966 (COX-2 Inhibitor), iloprost, methotrexate, thalidomide and thalidomide-related drugs (e.g., CELGEN), leflunomide, tranexamic acid, T-614, prostaglandin E1, tenidap, naproxen, meloxicam, ibuprofen, piroxicam, diclofenac, indomethacin, sulfasalazine, azathioprine, ICE inhibitors (inhibitors of the enzyme interleukin-1l converting enzyme), zap-70 and/or lck inhibitors (inhibitor of the tyrosine kinase zap-70 or lck), corticosteroid anti-inflammatory drugs (e.g., SB203580), TNF-convertase inhibitors, interleukin-17 inhibitors, gold, penicillamine, chloroquine, hydroxychloroquine, chlorambucil, cyclophosphamide, cyclosporine, total lymphoid irradiation, anti-thymocyte globulin, CD5-toxins, lobenzarit disodium, cytokine regulating agents (CRAs) HP228 and HP466, prednisone, orgotein, glycosaminoglycan polysulfate, minocycline, anti-IL2R antibodies, auranofin, phenylbutazone, meclofenamic acid, flufenamic acid, intravenous immune globulin, zileuton, mycophenolic acid (RS-61443), tacrolimus (FK-506), sirolimus (rapamycin), amiprilose (therafectin), cladribine (2-chlorodeoxyadenosine), azaribine, and methotrexate.

Examples of cardiac agents include, but are not limited to, quinapril, perindopril, nifedipine, ramipril, hydralazine, aspirin, ticagrelor, captopril, carvedilol, losartan, isosorbide dinitrate, valsartan, prasugrel, isosorbide mononitrate, eplerenone, and isosorbide dinitrate.

In a further aspect, the antimicrobial infection is a bacterial infection. Examples of bacterial infections include, but are not limited to, M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neisseria meningitidis, Neisseria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species. In a still further aspect, the bacterial infection is Staphylococcus aureus.

In a further aspect, the antimicrobial infection is a fungal infection. Examples of fungal infections include, but are not limited to, ringworm, a Candida infection, a fungal nail infection, Blastomyces infection, Cryptococcus gattii infection, Paracoccidioides infection, Coccidioides infection, and Histoplasmosis infection.

In a further aspect, the disease or disorder is a viral infection. Examples of viral infections include, but are not limited to, human immunodeficiency virus (HIV), human papillomavirus (HPV), herpes simplex virus (HSV), human cytomegalovirus (HCMV), chicken pox, infectious mononucleosis, mumps, measles, rubella, shingles, ebola, viral gastroenteritis, viral hepatitis, viral meningitis, human metapneumovirus, human parainfluenza virus type 1, parainfluenza virus type 2, parainfluenza virus type 3, respiratory syncytial virus, viral pneumonia, Chikungunya virus (CHIKV), Venezuelan equine encephalitis (VEEV), dengue (DENV), influenza, West Nile virus (WNV), human coronavirus, and zika (ZIKV).

In a further aspect, the disease or disorder is cancer. Examples of cancers include, but are not limited to, a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma).

In a further aspect, the disease or disorder is a cardiovascular disease. Examples of cardiovascular diseases include, but are not limited to, heart failure and myocardial infarction.

In a further aspect, the formulation and the agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

### G. REFERENCES

Bentley, R., A fresh look at natural tropolonoids. Nat Prod Rep, 2008. 25(1): p. 118-38.
Morita, Y., Matsumura, E., Okabe. T., Fukui. T., Ohe, T., Ishida, N., Inamori. Y. (2004) Biological activity of beta-dolabrin, gamma-thujaplicin, and 4-acetyltropolone, hinokitiol-related compounds, Biol Pharm Bull. 27, 1666-9.
Fotopoulou, T., et al., Antimicrobial/Antibiofilm Activity and Cytotoxic Studies of beta-Thujaplicin Derivatives. Arch Pharm (Weinheim), 2016. 349(9): p. 698-709.
Arima, Y., Antibacterial effect of beta-thujaplicin on staphylococci isolated from atopic dermatitis: relationship between changes in the number of viable bacterial cells and clinical improvement in an eczematous lesion of atopic dermatitis. Journal of Antimicrobial Chemotherapy, 2002. 51(1): p. 113-122.
Trust T J. (1975) Antibacterial activity of tropolone, Antimicrob Agents Chemother. 7, 500-6.
Ireland, P. J., et al., Synthetic alpha-Hydroxytropolones Inhibit Replication of Wild-Type and Acyclovir-Resistant Herpes Simplex Viruses. Antimicrob Agents Chemother, 2016. 60(4): p. 2140-9.
Lu, G., et al., Hydroxylated Tropolones Inhibit Hepatitis B Virus Replication by Blocking the Viral Ribonuclease H Activity. Antimicrob Agents Chemother, 2015. 59(2): p. 1070-1079.
Hu, Y., et al., beta-Thujaplicinol inhibits hepatitis B virus replication by blocking the viral ribonuclease H activity. Antiviral Res, 2013. 99(3): p. 221-9.
Tavis, J. E., et al., The hepatitis B virus ribonuclease His sensitive to inhibitors of the human immunodeficiency virus ribonuclease H and integrase enzymes. PLoS Pathog, 2013. 9(1): p. e1003125.
Donlin, M. J., et al., Troponoids Can Inhibit Growth of the Human Fungal Pathogen Cryptococcus neoformans. Antimicrob Agents Chemother, 2017. 61(4).
Shih, Y. H., et al., In vitro antimicrobial and anticancer potential of hinokitiol against oral pathogens and oral cancer cell lines. Microbiol Res, 2013. 168(5): p. 254-62.
Zhao, J., Plant troponoids: chemistry, biological activity, and biosynthesis. Curr Med Chem, 2007. 14(24): p. 2597-621.
Cao, F., et al., Synthesis and Evaluation of Troponoids as a New Class of Antibiotics. ACS Omega, 2018. 3(11): p. 15125-15133.
Tacconelli, E., M. Tumbarello, and R. Cauda. Staphylococcus aureus infections. N Engl J Med. 1998; 339(27): 2026-7.
Sakr, A., et al., Staphylococcus aureus Nasal Colonization: An Update on Mechanisms, Epidemiology, Risk Factors, and Subsequent Infections. Front Microbiol, 2018. 9: p. 2419.
Perichon, B. and P. Courvalin, VanA-type vancomycin-resistant Staphylococcus aureus. Antimicrob Agents Chemother, 2009. 53(11): p. 4580-7.
Nannini, E., B. E. Murray, and C. A. Arias, Resistance or decreased susceptibility to glycopeptides, daptomycin, and linezolid in methicillin-resistant Staphylococcus aureus. Curr Opin Pharmacol, 2010. 10(5): p. 516-21.
Morales, G., et al., Resistance to linezolid is mediated by the cfr gene in the first report of an outbreak of linezolid-resistant Staphylococcus aureus. Clin Infect Dis, 2010. 50(6): p. 821-5.
Hanslick, J. L., et al., Dimethyl sulfoxide (DMSO) produces widespread apoptosis in the developing central nervous system. Neurobiol Dis, 2009. 34(1): p. 1-10.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### H. EXAMPLES

Recently, over 100 troponoids were screened and evaluated for their antibacterial activities against Staphylococcus aureus, Escherichia coli, Acinetobacter baumannii, and Pseudomonas aeruginosa. Based on this extensive in vitro antimicrobial testing and biocompatibility testing, a novel non-toxic troponoid compound has been identified (compound no. 285, FIG. 1), which can inhibit eighteen methicillin-resistant MRSA strains from the St. Louis VA Healthcare System with similar MIC values as against a reference (S. aureus ATCC 25923) strain. Compound 285 can inhibit S. aureus at MIC less than 5 μg/mL with CC₅₀ higher than 100 μM on mammal cell lines (Cao, F. et al. Synthesis and Evaluation of Troponoids as a New Class of Antibiotics. ACS omega 3, 15125-15133 (2018)). A time-killing study showed 4×MIC compound 285 could completely kill S. aureus after 5-8 hours incubation in vitro (Cao, F. et al. Synthesis and Evaluation of Troponoids as a New Class of Antibiotics. ACS omega 3, 15125-15133 (2018)). This opens new avenues for development of this novel troponoid compound 285 as a new antibiotic to address the critical need to combat MDR bacterial infections, especially related to S. aureus. However, compound 285, similar to some other troponoid compounds, is not soluble in water but can be dissolved in the organic polar solvent dimethyl sulfoxide (DMSO), the most commonly used solvent for hydrophobic substances in biological experiments. It is known that DMSO can induce reported side effects including headaches, burning and itching on contact with the skin, and strong allergic reactions. DMSO is also a known developmental neurotoxin which may cause brain degeneration at level as low as 0.3 mL/kg and dampen respiration (Bakar, B. et al. Evaluation of the neurotoxicity of DMSO infused into the carotid artery of rat. Injury 43, 315-322 (2012); Takeda, K., Pokorski, M., Sato, Y., Oyamada, Y. & Okada, Y. Respiratory Toxicity of Dimethyl Sulfoxide. Adv. Exp. Med. Biol. 885, 89-96 (2016)).

To overcome the solubility limitation of novel troponoid antibacterial compound 285, a liposomal nanoparticle formulation of compound 285 has been developed (Cheng, X. & Cao, F. Liposomal Troponoid Compound Formulation, U.S. Provisional Patent Application No. 62/894,187, Filed on Aug. 30, 2019). Without wishing to be bound by theory, this formulation takes advantage of the great ability of liposomes to load both hydrophilic drugs (in the hydrophilic core of liposome) and hydrophobic drugs (via intercalation in the hydrophobic lipid bilayers). The novel liposomal 285 can eliminate the organic solvent such as DMSO from the final drug formulation and provide biocompatible, water-dispersible, controlled deliverable antibiotics for future clinical use in fighting MDRSA.

Here, formulations of troponoid compounds into liposomes, which are biocompatible and non-toxic, are described. The final formulation has almost no DMSO or residual chloroform and methanol, thus reducing the potential toxicity associated with these organic solvents. The liposome formulation not only helps to overcome the solubility limits of troponoid compound, but also provides a controlled/sustained-release mechanism for the troponoid drugs, whether they are hydrophobic or hydrophilic.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

1. Formation of a Liposome Formulation with No Drug (Water Only)

Soy Phosphatidylcholine (PC, SKU 840054P-200 mg from Avanti Polar Lipids) was dissolved in CHCl₃:MeOH (1:1) mixture at 50 mg/mL. Cholesterol (CH) was dissolved in a CHCl₃: MeOH (1:1) mixture at 30 mg/mL. α-tocopherol (Vitamin-E) was dissolved in the same solvent at 29 mg/mL. Using adjustable volume pipettes, a lipid mixture which consists of 25 mg PC, 3 mg of CH and 2 mg V-E was placed in a glass vial. A lipid cake (thin film) was formed inside the glass vial after blowing of N₂ gas to remove the CHCl₃ and MeOH. The glass vial containing the lipid cake was further dried in a vacuum oven for at least 6 hr. to remove any residual CHCl₃ and MeOH. This lipid cake was hydrated with 1 mL ultrapure water and vortexed and sonicated to form a homogenous solution. Then this 1 mL lipid-water mixture was extruded using a mini-extruder following standard extrusion procedure (e.g., through 0.2 μm 7 times and 0.1 μm PC membrane 7 times). This liposome water solution was used as a liposome control.

2. Formation of a Liposome Formulation of Troponoid Compound #62

Drug compound #62 was dissolved in DMSO at a concentration of 10 mM (e.g., 22.6 mg of compound 62 in 10 mL DMSO). Next, the drug solution was used to hydrate the same lipid cake in the glass vial prepared similarly as shown in Example 1 above. For example, 290 μL of 10 mM compound 62 in DMSO was added to the lipid cake, then 210 μL DMSO and 500 μL water was added to further hydrate the lipid cake. The lipid cake was vortexed and sonicate to form a homogenous solution. Then this 1 mL lipid-drug mixture was extruded using a mini-extruder following standard extrusion procedure (e.g., through 0.2 μm 7 times and 0.1 μm PC membrane 7 times). The formed nano sized liposome has a nominal drug concentration of 2.9 mM. The formed liposome can be further purified through dialysis (e.g. using Spectra/Por® Cellulose Ester (CE) dialysis membranes with molecular weight cut off of 100) against a large amount of water. This will remove the residual organic solvent DMSO (Mw=78) while retain the drug compound #62 (Mw=262).

3. Formation of a Liposome Formulation of Troponoid Compound #285

Drug compound #285 was dissolved in DMSO at 10 mM (e.g., 25.63 mg of compound 285 dissolved in 10 mL DMSO). To the glass vial containing the dried lipid cake similar as shown in example 1, 340 μL of compound 285 solution was added, then 640 μL of DMSO and 1 mL of water was added to further hydrate the lipid cake. The lipid-drug mixture was vortexed and sonicated to form a homogenous solution. Then this 2 mL lipid-drug mixture was extruded using a mini-extruder following standard extrusion procedure (e.g., through 0.2 μm 7 times and 0.1 μm PC membrane 7 times). This resulted in formation of 2 mL liposome with compound 285 at a nominal concentration of 1.7 mM for each mL of liposome solution. The formed liposome can be used directly or can be further purified through dialysis (e.g., using Spectra/Por® Cellulose Ester (CE) dialysis membranes with molecular weight cut off of 100) against a large amount of water. This will remove the residual organic solvent DMSO (Mw=78) while retain the drug compound #285 Mw=256.3).

4. Formation of a Liposome Formulation of Ceftriaxone

Drug compound Ceftriaxone was dissolved in DMSO at 10 mg/mL. To the glass vial containing the dried lipid cake similar as shown in Example 1 above, 1 mL solution was added to further hydrate the lipid cake. The lipid-drug mixture was vortexed and sonicated to form a homogenous solution. Then this 1 mL lipid-drug mixture was extruded using a mini-extruder following standard extrusion procedure (e.g., through 0.2 μm 7 times and 0.1 μm PC membrane 7 times). This result in formation of 1 mL liposome with Ceftriaxone at a nominal concentration of 10 mg/mL for each mL of liposome solution. The formed liposome can be used directly or can be further purified through dialysis (e.g., using Spectra/Por® Cellulose Ester (CE) dialysis membranes with molecular weight cut off of 100) against a large amount of water. This will remove the residual organic solvent DMSO (Mw=78) while retain the drug compound (Mw=555).

5. Formation of a Liposome Formulation of Gentamicin

Drug compound Gentamicin was dissolved in DMSO at 10 mg/mL. However, there was some solid remaining in this vial, suggesting that DMSO is not a good solvent for gentamicin. To the glass vial containing the dried lipid cake similar as shown in Example 1 above, 1 mL solution was added to further hydrate the lipid cake. 1 mL extra water was also added to further solubilize the drug. The lipid-drug mixture was vortexed and sonicated to form a homogenous solution. Then this 2 mL lipid-drug mixture was extruded using a mini-extruder following standard extrusion procedure (e.g., through 0.2 μm 7 times and 0.1 μm PC membrane 7 times). This resulted in formation of 1 mL liposome with Gentamicin at a nominal concentration of 5 mg/mL for each mL of liposome solution. The formed liposome can be used directly or can be further purified through dialysis (e.g., using Spectra/Por® Cellulose Ester (CE) dialysis membranes with molecular weight cut off of 100) against a large amount of water. This will remove the residual organic solvent DMSO (Mw=78) while retain the drug compound (Mw=478).

6. Formation of a Liposome Formulation of Hydrophobic Troponoid Compound Using the Intercalation Method without any DMSO

The example here shows an example of one hydrophobic troponoid compound, but can be applied to other hydrophobic troponoid compounds. 1 mg of compound #285, 25 mg soy PC, 3 mg cholesterol, and 2 mg V-E was dissolved in 5 mL CHCl₃:MeOH (1:1 v:v) in a glass vial. A lipid cake with trapped compound 285 (thin film) was formed inside the glass vial after blowing of N₂ gas to remove the CHCl₃ and MeOH. The glass vial containing the lipid cake was further dried in a vacuum oven for at least 6 hr. to remove any residual CHCl₃ and MeOH. This lipid cake was hydrated with 1 mL ultrapure water and vortexed and sonicated to form a homogenous solution. Then this 1 mL lipid-water mixture was extruded using a mini-extruder following standard extrusion procedure (e.g., through 0.2 μm 7 times and 0.1 μm PC membrane 7 times). This liposome water solution contains compound 285, which was intercalated in the lipid bi-layers.

7. Antimicrobial Efficacy of Prepared Liposome-Drug Samples

The Disk Diffusion Susceptibility Testing (Kirby-Bauer Method) was performed against wild-type (WT) S. aureus (ATCC 25923) and two Vancomycin Intermediate Staphylococcus aureus (VISA) strains, AR219 and 228, on the 5 formulated antibiotics/compounds shown in Examples 1-5 above. AR219 and AR228 was provided by Central Disease Control and Prevention (CDC). Both strains were multidrug-resistant including cefoxitin, clindamycin, erythromycin, gentamicin, levofloxacin, linezolid, mupirocin, oxacillin, penicillin, rifampin, and tetracycline. 0.034 μM (8.7 μg for compound 285) compound 62, 285, liposomal 62, 285, gentamicin, ceftriaxone and 20 μL liposome H₂O control was loaded onto a blank disk and sit in room temperature for 20 minutes. 1.5×10⁸ cfu/mL overnight bacteria was inoculated onto the Muller Hinton agar (MHA) plate. The disks were then loaded on to the plates. For 2% Mupirocin ointment, about 3-5 mm cream was loaded onto a blank disk, and then the disk was transported to the MHA plate immediately. The MHA plate was incubated 16-24 hours at 35±2° C. The diameter of each zone (including the diameter of the disc) was measured and recorded in mm.

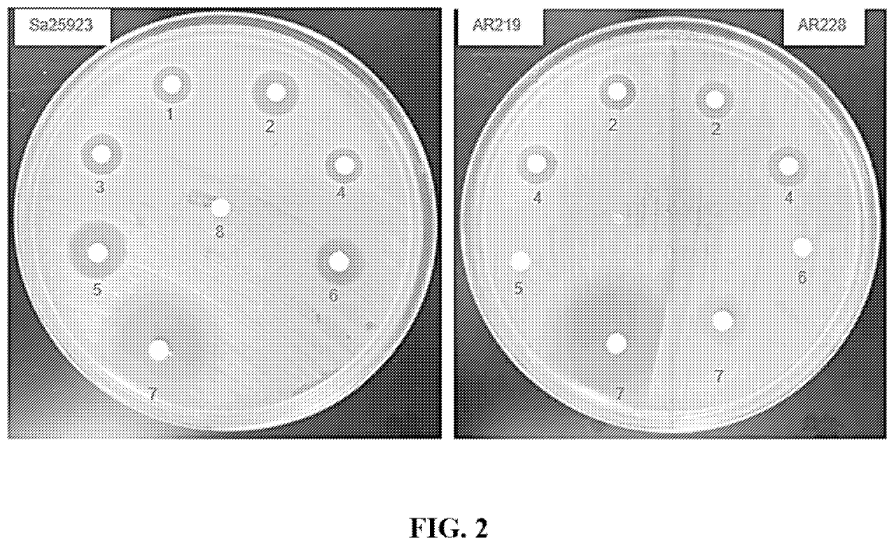

As shown in Table 1 and FIG. 2, liposomal control (L-H₂O in Table 1, disk 8 in FIG. 2) had no inhibitory effect on WT S. aureus strains (Sa25923). Compound 285 (285 in Table 1, and disk 1 in FIG. 2) had the similar inhibition zone against WT compare to liposomal 285 (L-285 in Table 1, and disk 2 in FIG. 2). Compound 62 (L-62 in Table 1, disk 3 in FIG. 2) also had similar inhibition zone as that for liposomal 62 (L-62 in Table 1, disk 4 in FIG. 2). In comparison, liposomal gentamicin (L-GEN in Table 1, disk 5 in FIG. 2) could inhibit WT strain, but had no inhibition on AR219 strain, consistent with antimicrobial susceptibilities testing (AST) result. Liposomal ceftriaxone (L-CRO in Table 1, disk 6 in FIG. 2) could inhibit WT with the similar inhibition zone as L-285 and L-62, but had no inhibition on AR228 strain, consistent with the AST result (ceftaroline resistant). 2% mupirocin ointment (MUP in Table 1, disk 7 in FIG. 2) could inhibit WT and AR219 strain, while it had no inhibition on AR228 strain, consistent with AST result.

TABLE 1

| | Antimicrobial susceptibility testing pattern (AST) | | | | | | | | |
|---|---|---|---|---|---|---|---|---|---|
| Bacteria | FOX | CLI | DAP | ERY | GEN | LVX | LZD | MUP | OXA |
| WT | Neg | S | S | S | S | S | S | | S |
| AR219 | R | R | NS | R | R | R | S | — | R |
| AR228 | R | R | NS | R | S | R | S | R | R |

R: resistance; S: sensitive; I: intermediate; NS: nonsusceptible; L: Liposomal
Cefoxitin (FOX); Clindamycin (CLI); Daptomyxin (DAP; Erthromycin (ERY); Gentamicin
(GEN); Levofloxacin (LVX); Linezolid (LZD); Mupirocin (MUP); Oxacillin (OXA);
Penicillin (P); Rifampin (RIF); Tetracycline (TET); Trimethoprim-sulfamethoxazole (SXT);
Vancomycin (VAN); Ceftriaxone (CRO).

| | Antimicrobial susceptibility testing | | | | | | | | |
|---|---|---|---|---|---|---|---|---|---|
| | pattern (AST) | | | | | Inhibition zone (mm) | | | |
| Bacteria | P | RIF | TET | SXT | VAN | 285 | L-285 | 62 | L-62 |
| WT | | S | S | S | S | 15.5 | 16.2 | 16 | 15.1 |
| AR219 | R | R | R | S | I | | 15.4 | | 15.4 |
| AR228 | R | S | S | S | I | | 15.4 | | 15.5 |

R: resistance; S: sensitive; I: intermediate; NS: nonsusceptible; L: Liposomal
Cefoxitin (FOX); Clindamycin (CLI); Daptomyxin (DAP; Erthromycin (ERY); Gentamicin
(GEN); Levofloxacin (LVX); Linezolid (LZD); Mupirocin (MUP); Oxacillin (OXA);
Penicillin (P); Rifampin (RIF); Tetracycline (TET); Trimethoprim-sulfamethoxazole (SXT);
Vancomycin (VAN); Ceftriaxone (CRO).
Inhibition zone (mm)

| Bacteria | L-GEN | L-CRO | MUP | L-H2O |
|---|---|---|---|---|
| WT | 21 | 16.6 | 31 | <=6 |
| AR219 | <=6 | | 31.4 | <=6 |
| AR228 | | <=6 | <=6 | <=6 |

Gentamicin (GEN); Mupirocin (MUP); Ceftriaxone (CRO).

From these results, it is seen that all three tested antibiotics showed the selective inhibition on the MDRSA strains; however, compounds 62 and 285 had a similar inhibition zone size for all three strains, indicating compounds 62 and 285 has a distinct working mechanism from the current front-line antibiotics. Meanwhile, liposome is highly biocompatible and liposome formulated drug, especially liposomal compound 285 (disk 2 in FIG. 2), can be effective in killing all three strains of S aureus, including MDRSA.

I. Additional Examples

1. Develop, Fabricate, and In Vitro Evaluate Novel Liposome-Loaded Drug Formulations of Synthetic Troponoid Compound 285.

Liposome preparation without DMSO. The liposome formulation will be further optimized to improve the drug loading, enhanced intranasal mucosa adhesion, and to investigate intravenous (i.v.) application. The incorporation of PEG may extend the liposome circulation by reducing uptake by the reticuloendothelial system (Deng, L. et al. Comparison of anti-EGFR-Fab′ conjugated immunoliposomes modified with two different conjugation linkers for siRNa delivery in SMMC-7721 cells. Int. J. Nanomedicine 8, 3271-3283 (2013)). Positively charged liposomes may markedly enhanced the adhesion of liposomes to the nasal mucosa (Iwanaga, K. et al. Usefulness of liposomes as an intranasal dosage formulation for topical drug application. Biol. Pharm. Bull. 23, 323-326 (2000)). Thus, the lipid compositions shown in Table 2 will be fabricated and investigated. Briefly compound #285, soy PC without and with stearyl amine (SA, positively charged), CH, and V-E or 1,2-Distearoyl-sn-Glycero-3-Phosphoethanolamine with conjugated methoxyl poly(ethylene glycol) (DSPE-mPEG) will be dissolved in 5 mL CHCl₃:MeOH (1:1 v:v) in each glass vial. Lipid cakes with trapped compound 285 (thin film) will form inside the glass vial after blowing of N₂ gas to remove the CHCl₃ and MeOH. The glass vial containing the lipid cake will be further dried in a vacuum oven for at least 6 hr to remove any residual CHCl₃ and MeOH. This lipid cake will be hydrated with 1 mL 0.9% NaCl solution and vortexed and sonicated to form a homogenous solution. Then this 1 mL lipid-water mixture will be extruded using a mini-extruder following standard extrusion procedure (e.g., through 0.2 μm 7 times and 0.1 μm PC membrane 7 times). These liposome aqueous formulations will contain hydrophobic compound 285 intercalated in the lipid bi-layers. A similar liposome preparation for another hydrophobic drug (simvastatin) at 65 mg/mL (or 6.5 wt %) was previously demonstrated (Cheng, X. et al. A liposomal statin formulation, U.S. patent application Ser. No. 15/949,000. (2018)).

In vitro characterization and quantification of liposomes for drug loading and release. Particle size and charge (Zeta potential) of the above liposomes will be quantified by ZetaPals (Brookhaven labs, USA). Non-encapsulated drug and encapsulated liposome will be separated by size exclusion chromatography (SEC) using Sephadex G-50 beads. Morphology of liposomes will be examined using cryo-Transmission electron microscopy. Drug loading inside the liposome will be quantified using High Performance Liquid Chromatography (HPLC), as described herein (Liposome Technology (Third Edition): Entrapment of Drugs and Other Materials into Liposomes, Edited by Gregory Gregoriadis. (Informa HealthCare)). Drug release will follow the publication (Cheng, X. et al. Comparison of Two Nanoparticle Formulations for Localized Delivery of Platelet-Derived Growth Factor (PDGF) from Aligned Collagen Fibers. Pharm. Nanotechnol. 1, 105-114 (2013)).

Lyophilization, Reconstitution, and Dosage confirmation study. Sucrose will be chosen as a cryoprotectant in the lyophilization process (Kannan, V., Balabathula, P., Thoma, L. A. & Wood, G. C. Effect of sucrose as a lyoprotectant on the integrity of paclitaxel-loaded liposomes during lyophilization. J. Liposome Res. 25, 270-278 (2015); Chen, Y. et al. A lyophilized sterically stabilized liposome-containing docetaxel: in vitro and in vivo evaluation. J. Liposome Res. 27, 64-73 (2017)). The optimal ratio of cryoprotectant-to-lipid (C/L) mass ratio will be determined by investigating the size changes before and after lyophilization and reconstitution of three different C/L ratios (e.g., 7:1, 8:1, and 9:1). Liposome will be reconstituted by adding sterile saline (0.9%), brief vortexing and sonication and followed by filtration through 5 μm sterile syringe filter. The dosage of compound 285 in the lyophilized liposome vial and the reconstituted liposomes will both be analyzed using HPLC. 2% DMSO in acetonitrile will be used to extract the liposome. The chromatographic separation of lipid and compound 285 will be performed using Chromolith Performance RP 18e column (Sigma). The mobile phase composed of 5 mM ammonium acetate, methanol and acetonitrile and a gradient elution program will be used (Deshpande, N. M., Gangrade, M. G., Kekare, M. B. & Vaidya, V. V. Determination of free and liposomal Amphotericin B in human plasma by liquid chromatography-mass spectroscopy with solid phase extraction and protein precipitation techniques. J. Chromatogr. B 878, 315-326 (2010)).

Antimicrobial testing of the above liposome-drug formulations. Compound 285 in DMSO as positive control, liposomal 285 formulations, and corresponding liposome controls will be tested using the same method as shown in FIG. 2. The diameter of each zone (including the diameter of the disc) will be measured and recorded in mm.

MTS cytotoxicity assays (CC₅₀). A549 cells will be seeded in 96-well plates and incubated in Dulbecco's Modified Eagle Medium (DMEM). The formulated drug will be diluted in the medium to the indicated concentrations and added to the cells 48 h after plating, with each concentration tested in triplicate. Soluble MTS reagent [3-(4,5-dimethylthiazol-2-yl)-5-(3-carboxymethoxyphenyl)-2-(4-sulfophenyl)-2H-tetrazolium, Promega] will be added 72 h after incubation, after incubation for 90 min., and absorbance will be read at 490 nm. The absorbance interference of nanoparticle at 490 nm will be excluded using a previously described method (Cheng, X. & Kuhn, L. Chemotherapy drug delivery from calcium phosphate nanoparticles. Int. J. Nanomedicine 2, 667-674 (2007)). The CC₅₀ is calculated as the concentration of inhibitor required to reduce cell viability 50% relative to untreated cells. The data are plotted as log[inhibitor] versus response and fit to a variable slope model using Graph Pad Prism.

Without wishing to be bound by theory, it is expected that biocompatible liposomes with controlled charges (+, −, or close to neutral) and acceptable antimicrobial properties (e.g., Zone of inhibition comparable to drug in DMSO) can be fabricated and further selected for intranasal evaluation as detailed below or for other future study (e.g., i.v. route). The lipid composition is the major determining factor for encapsulation efficiency and characteristics of liposomes. In the case that the proposed formulation outlined in Table 2 cannot lead to stable liposomes or unexpected toxic, alternative lipid compositions will be investigated by adjusting the exact amounts, or by using alternative lipids such as dipalmitoylphosphatidylcholine (DPPC) which was investigated for intranasal application (Iwanaga, K. et al. Usefulness of liposomes as an intranasal dosage formulation for topical drug application. Biol. Pharm. Bull. 23, 323-326 (2000)).

2. Perform Proof-of-Principle Animal Studies to Identify the Top Safe and Effective Drug Formulations for Phase II SBIR Development.

Colonization with S. aureus in the anterior nares is considered the most important factor of subsequent invasive S. aureus infections, particularly for those requiring operation procedures, hemodialysis, implanted devices, or treatment in intensive care units. According to the CDC, about one in three (33%) people carry S. aureus bacteria in their nose, usually without any illness. About two in every 100 people carry MRSA (How common is MRSA? https://www.cdc.gov/mrsa/healthcare/index.html, Last access date 08/16/2019). Clearance of S. aureus nasal colonization can reduce the subsequent risk of developing S. aureus infections. Currently, Bactroban Nasal (2% Mupirocin calcium ointment) remains the standard topical agent at eradicating nasal S. aureus. However, mupirocin resistance is emerging in MDR S. aureus (van Rijen, M., Bonten, M., Wenzel, R. & Kluytmans, J. Mupirocin ointment for preventing Staphylococcus aureus infections in nasal carriers. Cochrane database Syst. Rev. CD006216 (2008). doi:10.1002/14651858.CD006216.pub2). Thus, for in vivo study, a nasal bacterial infection and intranasal drug treatment model will be used to test the disclosed liposome formulation. Intranasal route of administration of drugs has the advantages of non-invasive and painless. Results testing another liposomal formulation using the proposed intranasal route in swines were previously published (Dhakal, S. et al. Liposomal nanoparticle-based conserved peptide influenza vaccine and monosodium urate crystal adjuvant elicit protective immune response in pigs. Int. J Nanomedicine 13, 6699-6715 (2018)).

Liposomal compound 285 in vivo toxicity study in mice without bacterial infection. For the toxicity study, four positively charged, reconstituted, liposome formulations with the same drug loading (e.g., dosage at 25 mg/mL after reconstitution with saline) will be evaluated, since it is known that positively charged liposomes are more toxic than negatively charged ones (Knudsen, K. B. et al. Differential toxicological response to positively and negatively charged nanoparticles in the rat brain. Nanotoxicology 8, 764-774 (2014)), but they are better for nasal mucosal adhesion (Iwanaga, K. et al. Usefulness of liposomes as an intranasal dosage formulation for topical drug application. Biol. Pharm. Bull. 23, 323-326 (2000)). BALB/c mice of both sexes will be used. On days 1 and 3, each mouse (anesthetized with isoflurane) will be held in an upright position and the reconstituted liposome solution will be administrated (4 μl per nostril using a micropipette tip, dosage: 5 mg/kg). Four cationic liposomal drug formulations and one saline control will be evaluated (5 groups total). Each group will have 4 female and 4 male mice, total 40 mice. Seven days after the second compound administration, mouse nasal cavities will be dissected for mucosal toxicity evaluation. The potential nasal mucosa toxicity, including potential damage to the olfactory system will be assessed on the basis of histopathological analysis of the recovered nose according to the reported methods (Chhibber, S., Gupta, P. & Kaur, S. Bacteriophage as effective decolonising agent for elimination of MRSA from anterior nares of BALB/c mice. BMC Microbiol. 14, 212 (2014); Fukuyama, Y. et al. Nasal Administration of Cholera Toxin as a Mucosal Adjuvant Damages the Olfactory System in Mice. PLoS One 10, e0139368 (2015); Parquet, M. D. C., Savage, K. A., Allan, D. S., Davidson, R. J. & Holbein, B. E. Novel Iron-Chelator DIBI Inhibits Staphylococcus aureus Growth, Suppresses Experimental MRSA Infection in Mice and Enhances the Activities of Diverse Antibiotics in vitro. Front. Microbiol. 9, 1811 (2018)). The sections will be picked on separate slides, stained with hematoxylin and eosin, and the slides then be examined under a microscope to evaluate the extent of damage, compared to control noses and olfactory epitheliums. Of the above four liposomal drug formulations, two formulations with the least toxicity to nose and olfactory epithelium will be chosen for the next efficacy study.

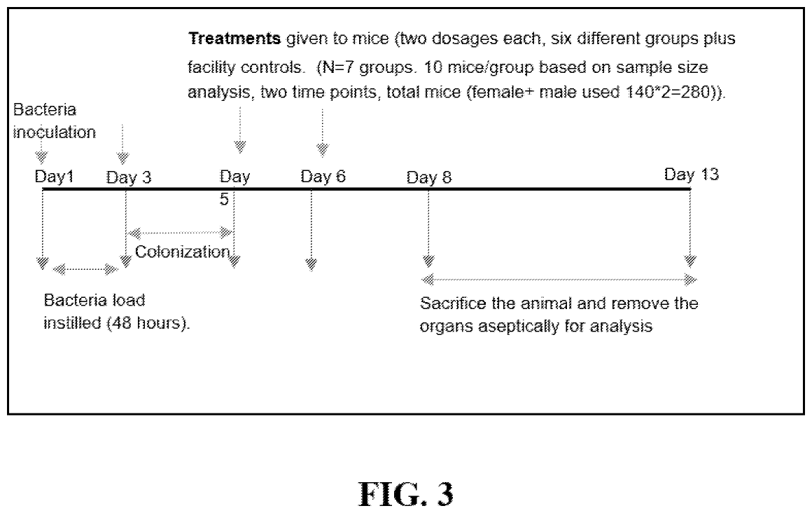

Liposomal compound 285 in vivo efficacy study in mice with MDRSA nasal infection. A male BALB/c mice nares colonization model first described by Chhibber et al. will be used (Chhibber, S., Gupta, P. & Kaur, S. Bacteriophage as effective decolonising agent for elimination of MRSA from anterior nares of BALB/c mice. BMC Microbiol. 14, 212 (2014)). S. aureus CDC #10 will replace S. aureus ATCC43300 for the purpose of MDRSA kill testing. Bacterial cultures will be grown in brain heart infusion (BHI) medium. Overnight cultures will be harvested and washed and re-suspended in sterile saline. Plate counting to confirm inoculum CFU/mL will be done on blood agar plate (TSA with sheep blood, Remel) (BAP). On day 1 and day 3, mice will be anesthetized using isoflurane and infection will be initiated by nasal instillation to mice held in an upright position to make sure bacterial suspension enters each nostril. Mice will be left for 48 hours to allow nasal colonization. On days 5 and 6, the mice will be anesthetized and treated as shown in FIG. 3. The following groups will be used: Group 1: Formulation 1 control (liposome only); Group 2: Liposomal 285 (e.g., reconstituted formulation 1 at 25 mg/mL drug loading, 4 μL per nostril); Group 3: Formulation 2 control (liposome only); Group 4: Liposomal 285 (e.g., reconstituted formulation 2 at 25 mg/mL drug loading, 4 μL per nostril); Group 5-7: Controls. Group 5: Saline treatment control; Group 6: Mupirocin treatment control (5 mg/kg dissolved in water; given once), and Facility control (without infection and any treatments).

On days 2 (day 8) and 7 (day 13) post-treatment, mice will be sacrificed, the area around the nose will be wiped with 70% alcohol and the entire nose along with the nasal bone structure will be excised using sterile scissors. Half the tissue from each mouse will be used for histopathological examination; the other half will to be homogenized for assays (below). Lungs and cervical lymph nodes will also be aseptically removed. The following testing will be performed on the removed organ/tissues:

Quantitative bacteriology. The mouse noses, lungs and lymph nodes will be homogenized in Tryptic soy broth (TSB). Aliquots (100 μl) of 10-fold serial dilutions of the homogenates will be cultured on BAP (with 20 μg/mL ampicillin pre-plating, note: ampicillin is broad spectrum but not kill S Aureus CDC #10) to quantify the number of viable S. aureus organisms in the respective organs. Plate counts of serial dilutions of the tissue homogenates will be made after 24 h growth at 35±2° C. The reduction ratio of MDRSA will be determined for each treatment group:

$$Bacteria survival ratio ( \% ) = \text{?} \times 100 ,$$

$$\text{?} \text{indicates text missing or illegible when filed}$$

where Nₛ stands for the number of MDRSA bacteria colonies for samples, and N_{con} stands for the number of bacteria colonies for the non-treated control.

Histopathological examination. Extent of injury caused by S. aureus [day 8 and healing (day 13)] of the colonized mouse nose following therapy with liposomal compound 285 or control will be assessed on the basis of histopathological analysis of the injured and recovered nose as described in the toxicology study.

Myeloperoxidase (MPO) estimation. The nose homogenates will be processed for MPO determination as per the reported method (Chhibber, S., Gupta, P. & Kaur, S. Bacteriophage as effective decolonising agent for elimination of MRSA from anterior nares of BALB/c mice. BMC Microbiol. 14, 212 (2014)). The absorbance is read immediately at 490 nm over a period of 4 minutes. MPO is calculated as the change in optical density (O.D) x dilution factor (D.F).

Cytokine and Chemokine assays. The levels of cytokines and chemokines in the nose homogenate supernatants will be measured using the 21-plex Milliplex MAP mouse cytokine/chemokine kits (Millipore, Ltd., Billerica, Mass.) on a Luminex MagPixsystem (Luminex, Austin, Tex.) as specified by the manufacturer. Samples will be assayed in duplicate, and cytokine/chemokine concentrations will be calculated against the standards using Beadview software.

Serum analysis. Blood will be drawn on days 1, 6, 8, and 13 and analyzed for serum antibody to Staphylococcal Toxic Shock Antigen according to reported ELISA assay (Ritz, H. L., Kirkland, J. J., Bond, G. G., Warner, E. K. & Petty, G. P. Association of high levels of serum antibody to staphylococcal toxic shock antigen with nasal carriage of toxic shock antigen-producing strains of Staphylococcus aureus. Infect. Immun. 43, 954-958 (1984)). In addition, serum will be analyzed for systemic drug concentration using HPLC as described above.

Intranasal administration of liposomal 285 will completely or significantly kill CDC #10 S. aureus strains. In contrast, mupirocin ointment will have minimal/no inhibition on CDC #10 S. aureus due to drug resistance. It is possible there are residual normal flora contamination such as Staphylococcus epidermidis, Corynebacterium sp. etc., which will affect the cell counting from homogenized tissues. This effect is reduced by using of ampicillin beta lactam antibiotic during quantitative bacteriology. If the problem still exists, S. aureus can be identified using morphological differences or “Matrix Assisted Laser Desorption/Ionization” to identify the bacteria (Kiser, K. B., Cantey-Kiser, J. M. & Lee, J. C. Development and characterization of a Staphylococcus aureus nasal colonization model in mice. Infect. Immun. 67, 5001-5006 (1999)).

3. Statistical Analysis

For comparisons of three or more numeric data sets, variances within the data will be compared with a Bartlett's test. If the variances are not significant, a one-way Analysis of Variance (ANOVA) will be performed accompanied by a Tukey's post-hoc test for group discrimination. If the variances are significantly different, a Kruskal-Wallis test will be performed accompanied by a Dunn's post-hoc test for group discrimination. Statistical significance will be set at p<0.05.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A liposome formulation comprising a troponoid and a lipid, wherein the troponoid has a structure represented by a formula: 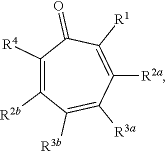
wherein R¹ is selected from hydrogen, halogen, —OH, —SH, —OC(O)Ar¹, —SC(O)Ar¹, —OC(O)(C1-C4 alkyl)Ar¹, —SC(O)(C1-C4 alkyl)Ar¹, and —OSO₂Ar¹;
wherein Ar¹, when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein each of R^{2a} and R^{2b} is independently selected from hydrogen, halogen, —OH, —CO₂H, and Ar²;
wherein Ar², when present, is C6-C12 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein each of R^{3a} and R^{3b} is independently selected from hydrogen, halogen, C1-C6 alkyl, —C(O)R¹¹, —C(O)Ar³, and Ar³;
wherein R¹¹, when present, is selected from C1-C4 alkyl and C3-C6 cycloalkyl;
wherein Ar³, when present, is selected from C2-C5 heteroaryl and C6-C12 aryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and
wherein R⁴ is selected from hydrogen, halogen, and —OH,
or a pharmaceutically acceptable salt thereof.

2. The formulation of claim 1, wherein R¹ is selected from hydrogen, —OH, and —OC(O)Ar¹.

3. The formulation of claim 1, wherein R¹ is —OC(O)Ar¹.

4. The formulation of claim 1, wherein each of R^{2a} and R^{2b} is hydrogen.

5. The formulation of claim 1, wherein each of R^{3a} and R^{3b} is hydrogen.

6. The formulation of claim 1, wherein R⁴ is hydrogen.

7. (canceled)

8. The formulation of claim 1, wherein the troponoid has a structure represented by a formula:

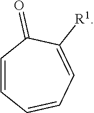

9. (canceled)

10. The formulation of claim 1, wherein the troponoid is selected from:

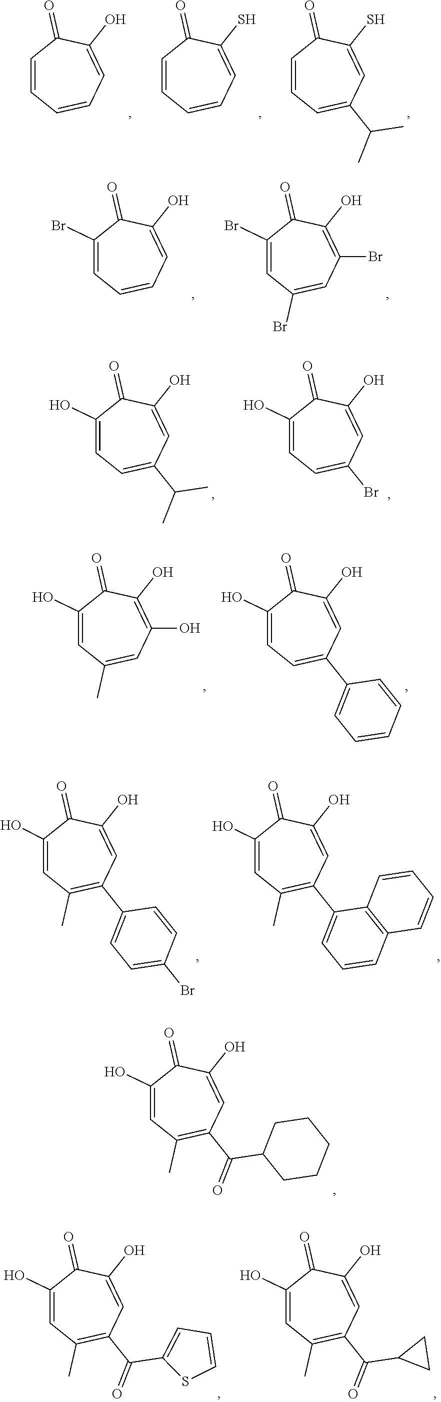

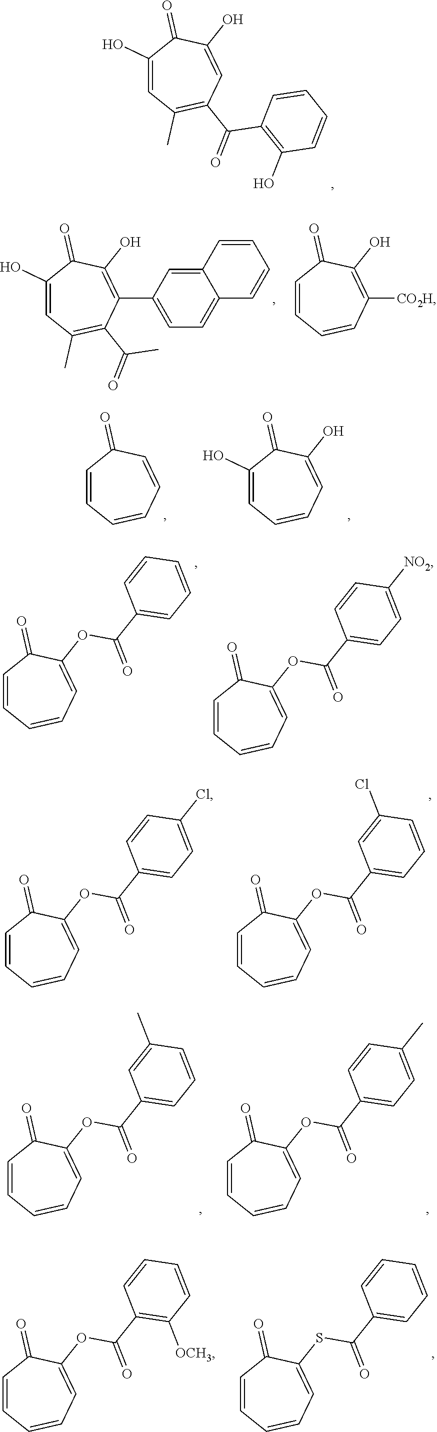

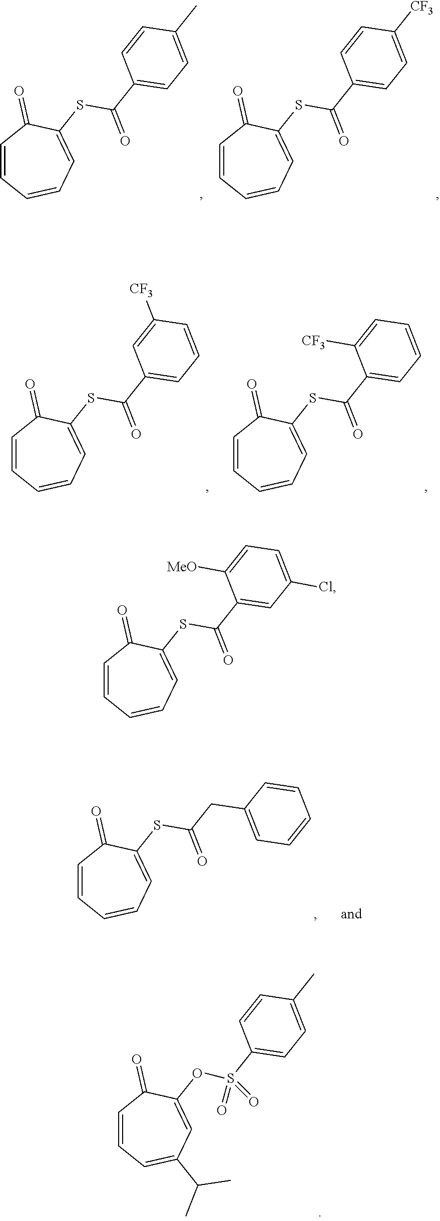

11. (canceled)

12. The formulation of claim 1, wherein the troponoid is selected from.

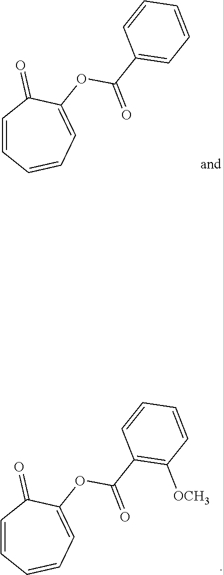 [SMILES: O=C(Oc1cccccc1=O)c1ccccc1 ; COc1ccccc1C(=O)Oc1cccccc1=O]

13-16. (canceled)

17. The formulation of claim 1, wherein the troponoid is present in an amount of about 15 wt % or less.

18. The formulation of claim 1, wherein the lipid is selected from phosphatidylcholine (PC), 1,2-dimyristoyl-sn-glycero-3-phosphocholine (DMPC), 1,2-dipalmitoyl-sn-glycero-3-phosphocholine (DPPC), and 1,2-distearoyl-sn-glycero-3-phosphocholine (DSPC).

19. The formulation of claim 1, wherein the lipid is phosphatidylcholine (PC).

20. The formulation of claim 1, wherein the lipid is present in an amount of from about 60 wt % to about 99 wt %.

21. The formulation of claim 1, wherein the lipid is sourced from soybeans or egg yolk.

22. The formulation of claim 1, further comprising one or more selected from cholesterol and vitamin E.

23-45. (canceled)

46. A nanoparticle comprising the formulation of claim 1.

47. A method for treating a disease or disorder in a subject, the method comprising administering to the subject an effective amount of the formulation of claim 1, wherein the disease or disorder is a viral infection, an antimicrobial infection, cancer, an inflammatory disease, or a cardiovascular disease.

48. The method of claim 47, wherein the disease or disorder is an antimicrobial infection.

49. The method of claim 48, wherein the antimicrobial infection is a bacterial infection.

50. (canceled)

51. The method of claim 49, wherein the bacterial infection is Staphylococcus aureus.

52-94. (canceled)
